(19) United States
(12) Patent Application Publication
(10) Pub. No.: US 2026/0284476 A1
(43) Pub. Date: Sep. 24, 2026

(54) METHODS FOR TREATING NEUROMUSCULAR DISEASES

(71) Applicant: Hoffmann-La Roche Inc., Little Falls, NJ (US)

(72) Inventors: Xing CHEN, Riechen (CH); Juergen GOTTOWIK, Basel (CH); Slawomir OPALKA, Lódz (PL); Maryam OSKOUI, Montreal (CA); David HERZIG, Allschwil (CH)

(21) Appl. No.: 19/476,647

(22) PCT Filed: Apr. 19, 2024
(86) PCT No.: PCT/EP2024/060819

§ 371 Date: Oct. 17, 2025

(30) Foreign Application Priority Data
Apr. 20, 2023 (EP) 23169021.5

(51) Int. Cl. A63B 24/00; A63B 71/06; G16H 20/30

(52) U.S. Cl. CPC A63B 24/0075; A63B 71/0622; G16H 20/30; A63B 2213/00; A63B 2220/05; A63B 2220/24; A63B 2220/803; A63B 2220/806

(57) ABSTRACT

The present invention relates to computer-implemented methods comprising: providing a user interface to a subject that has been diagnosed as having a neuromuscular disease or disorder and optionally undergoing treatment with a therapeutic compound or composition for the treatment of the neuromuscular disease or disorder; receiving movement data comprising coordinates of a plurality of joints of the subject or data derived therefrom, at a plurality of time points, and in response to receiving movement data satisfying a set of predetermined criteria, triggering a change in the user interface, wherein the change is specific to the set of predetermined criteria satisfied and wherein the set of predetermined criteria is selected from a plurality of sets of predetermined criteria, each set identifying a predetermined movement. Related systems and digital physiotherapy methods and methods for treating a subject with a neuromuscular disorder using the digital physiotherapy method are also described.

### CROSS REFERENCE TO RELATED APPLICATIONS

This application is a U.S. national stage application under 35 U.S.C. § 371 of International Application No. PCT/EP2024/060819, filed internationally on Apr. 19, 2024, which claims priority to European Application No. 23169021.5, filed on Apr. 20, 2023.

### FIELD OF THE INVENTION

The present invention relates to methods for treating a subject with a neuromuscular disease, using a digital physiotherapy tool. Related products for use in such methods are also described.

### BACKGROUND TO THE INVENTION

Spinal Muscular Atrophy (SMA) is an autosomal recessive and frequently severe neuromuscular disease characterized by the loss of a motor neurons resulting in progressive muscle atrophy. SMA is associated with mutations in the survivor motor neuron 1 (SMN1) gene. SMA prognosis has been transformed with the recent availability of a number of effective disease-modifying therapies. As rare disease therapies are implemented across health care systems, there is a further need for evidence regarding longer term real-world outcomes for children with SMA and regarding the co-interventions needed to optimize outcomes and patient experience.

Co-interventions to promote physical activity and active participation have remained unchanged for this population, with limited evidence to guide decisions on optimizing outcomes. Exercise has been shown to prolong survival in SMA mouse models and increase in full-length SMN protein transcription. Among children with neuromuscular disease, satisfaction with active participation is associated with increased quality of life, promotes self-concept and physical health, and is an important patient-centered outcome. However, children with disabilities are at risk of lower participation in leisure and fall far behind the Canadian physical activity recommendation of 60 minutes daily.

Therefore, there remains a need for improved therapeutic approaches for neuromuscular diseases.

### SUMMARY OF THE INVENTION

The inventors postulated that the use of active videogames for rehabilitation (exergaming) could help to promote physical activity in youth with neuromuscular disorders, in particular SMA, ensuring that these patients perform an appropriate amount of physical activity (i.e. delivering physiotherapy treatment in the appropriate dose for the patient). Exergaming is expected to have a positive impact on motivation towards training, is flexible in scheduling, and has proven useful to enhance strength, coordination, and mobility in other conditions. The inventors have developed a home-based digital physiotherapy tool (exergame) specifically for youth with neuromuscular disorders using the Microsoft® Kinect Azure sensor. The tool is expected to improve or enable sustained adherence to a physiotherapy programme and/or improve, stabilise or reduce the degradation of the subject's motor function.

Thus, according to a first aspect, the disclosure provides a computer-implemented method comprising: providing a user interface to a subject that has been diagnosed as having a neuromuscular disease or disorder and optionally undergoing treatment with a therapeutic compound or composition for the treatment of the neuromuscular disease or disorder; receiving movement data comprising coordinates of a plurality of joints of the subject or data derived therefrom, at a plurality of time points; and in response to receiving movement data satisfying a set of predetermined criteria, triggering a change in the user interface, wherein the change is specific to the set of predetermined criteria satisfied and wherein the set of predetermined criteria is selected from a plurality of sets of predetermined criteria, each set identifying a predetermined movement.

The method may have any one or more of the following optional features.

The method may further comprise, upon receiving the movement data, determining whether the movement data satisfies each of the plurality of sets of predetermined criteria.

The plurality of sets of predetermined criteria may comprise: a first set of predetermined criteria identifying an elbow flexion gesture, a second set of predetermined criteria identifying a horizontal abduction gesture, a third set of predetermined criteria identifying a head rotation gesture, a fourth set of predetermined criteria identifying a thoracic extension gesture, and a fifth set of predetermined criteria identifying an open-close hand gesture.

One or more of the predetermined criteria may apply to angles between predetermined vectors derived from one or more joints or to projections of angles between predetermined vectors on predetermined body planes. Each set of predetermined criteria may be individually selected from: (i) a set of criteria that apply to movement data at a single time point and (ii) a set of criteria comprising at least one criterion that applies at a first time point and at least one criterion that applies at a second time point. The second time point may be any time point subsequent to the first time point. The predetermined vectors may be selected from: a forearm vector, an upper arm vector, a chest vector, a head forward vector, a torso forward vector, an arm vector, a torso down vector, a torso up vector, a finger vector, and a palm vector.

At least one criterion may apply to an angle between two predetermined vectors derived from one or more joints or to the projection of an angle between two predetermined vectors on a predetermined body plane. The method may comprise calibrating the at least one criterion by recording movement data of the subject while the subject is performing the movement associated with the predetermined criterion and identifying an expected range of the angle or the angle projection while the subject is performing the movement associated with the predetermined criterion.

The plurality of sets of predetermined criteria may comprise a first set of predetermined criteria identifying an elbow flexion gesture. The first set of predetermined criteria may comprise a criterion that the angle between a forearm vector and an upper arm vector on the same side changes from a first value in a first predetermined range to a second value in a second predetermined range. Instead or in addition to this, the first set of predetermined criteria may comprise a criterion that the angle between a forearm vector and an upper arm vector on the same side is below a threshold value that is in the second predetermined range. The second predetermined range may be between 10 degrees and 50 degrees. The first predetermined range may be between 80 degrees and 120 degrees.

The plurality of sets of predetermined criteria may comprise a second set of predetermined criteria identifying a horizontal abduction gesture, comprising a first predetermined criterion that the angle between a torso forward vector and an arm vector is within a first predetermined range or below a first predetermined threshold value that is in said first predetermined range, at a first time point, and a second predetermined criterion that the angle between the torso forward vector and the arm vector is within a second predetermined range or above a second predetermined threshold value that is in the second predetermined range, at a second time point that is subsequent to the first time point. The first predetermined range may be between 0 and 70 degrees. The second predetermined range may be between 30 degrees and 80 degrees. a second set of predetermined criteria identifying a horizontal abduction gesture may further comprise a third predetermined criterion that an angle between a torso up vector and a forearm arm vector on the same side as that to which the first predetermined criterion applies is within a predetermined range at the first and/or second time points.

The plurality of sets of predetermined criteria may comprise a third set of predetermined criteria identifying a head rotation gesture, comprising a first predetermined criterion that the angle between a head forward vector and a torso forward vector at a first time point is within a first predetermined range or above a first predetermined threshold that is in said first predetermined range, and a second predetermined criterion that the angle between the head forward vector and the torso forward vector is within a second predetermined range or below a second predetermined threshold that is in said second predetermined range, at a second time point that is subsequent to the first time point. The first predetermined range may be 42±5 degrees. The second predetermined range may be 0±10 degrees.

The plurality of sets of predetermined criteria may comprise a fourth set of predetermined criteria identifying a thoracic extension gesture, comprising a first predetermined criterion that the angle between the left and right chest vectors at a first time point is within a first predetermined range or above a predetermined threshold value that is in the first predetermined range, and a second predetermined criterion that the angle between the left and right chest vectors is within a second predetermined range at a second time point that is subsequent to the first time point. The first predetermined range may be 190±12 degrees. The second predetermined range may be 160±15 degrees.

The plurality of sets of predetermined criteria may comprise a fifth set of predetermined criteria identifying an open-close hand gesture, comprising: a first predetermined criterion that an angle between a finger vector and a palm vector on the same side at a first time point is within a first predetermined range or below a first predetermined threshold value that is in the first predetermined range, and a second predetermined criterion that the angle between the finger vector and a palm vector is within a second predetermined range or above a second predetermined threshold value that is in the second predetermined range, at a second time point that is subsequent to the first time point. The first predetermined range may be between 30 degrees and 100 degrees. The second predetermined range may be between 100 degrees and 180 degrees. The fifth set of predetermined criteria identifying an open-close hand gesture may further comprise a third predetermined criterion that applies to an angle between a forearm vector and an upper arm vector on the same side as that which satisfies the first and second predetermined criteria. The third predetermined criterion may be defined as the angle changing between a first predetermined value and a second predetermined value for identifying an elbow flexion. The third predetermined criterion may be defined as the angle being at or below the second predetermined value for identifying an elbow flexion. A fifth set of predetermined criteria identifying an open-close hand gesture may further comprise a fourth predetermined criterion that applies to the distance between a hand tip joint and a wrist joint on the same side as that which satisfies the first and second predetermined criteria, optionally wherein the fourth predetermined criterion is defined as said distance being less than a predetermined ratio of a reference value for said distance.

The plurality of joints may include one or more or all of a plurality of locations that aligns with a subject's: left elbow, right elbow, left wrist, right wrist, left shoulder, right shoulder, head, neck, spine at chest level, spine at navel level, pelvis, left clavicle, right clavicle, left hand, right hand, left hand tip, and right hand tip.

All coordinates may be 3D coordinates. The movement data may have been acquired using one or more movement sensors selected from a 2D camera and a 3D camera.

According to a second aspect, there is provided a therapeutic compound or composition for use in a method of treating a neuromuscular disease or disorder in a subject, wherein the method comprises: administering a therapeutically effective amount of the therapeutic compound or composition to the subject, and implementing a digital physiotherapy method comprising the steps of any embodiment of the first aspect. The method may improve, stabilise or reduce the degradation in a motor function score in the subject. The method may improve, stabilise or reduce the degradation in a motor function score in the subject compared to a subject treated with a method comprising administering a therapeutically effective amount of the therapeutic compound or composition to the subject without implementing the digital physiotherapy method.

Embodiments of the present aspect may have any one or more of the following optional features.

The method may comprise implementing the digital physiotherapy method according to a predetermined digital physiotherapy dosage regimen. The predetermined physiotherapy dosage regimen may comprise the use of the digital physiotherapy tool for at least 15 to 25 minutes per day at least 4 times per week.

The method may increase the likelihood of continued administration of a physiotherapy dosage regimen. Continued administration of a physiotherapy dosage regimen may refer to adherence to the physiotherapy dosage regimen for at least 3 months, at least 6 months, at least 9 months or at least 12 months.

A motor function score may be selected from: the revised upper limb module (RULM), the Hammersmith Functional Motor Scale (HFMS), the Motor Function Measure (MFM), the Fatigue Severity Scale (FSS), the Modified Fatigue Impact Scale (MFIS), the Visual Analogue Scale for Fatigue (VAS-F), the Children's Assessment of Participation and Enjoyment (CAPE), the PedsQL™ 4.0 Generic Core Scales (Pediatric Quality of Life Inventory™), the PROMIS-SF, the SMAIS, the SMAIS-ULM, and the Canadian Occupational Performance Measure (COPM). The method may improve, stabilise or reduce a degradation in a motor function score after about 3 months, about 6 months, about 9 months or about 12 months of treatment, compared to a subject treated with a method comprising administering a therapeutically effective amount of the therapeutic compound or composition to the subject without implementing the digital physiotherapy method. The therapeutic compound or composition may be a disease modifying therapeutic. The therapeutic compound or composition may be selected from nusinersen and risdiplam. A degradation may be a reduction in score or an increase in score, depending on the score.

According to any embodiment of any aspect, the subject may have a neuromuscular disease or disorder selected from: a motor neuron disease, a toxic neuropathy, a congenital myopathy, a muscular dystrophy, and spinal muscular atrophy. The spinal muscular atrophy may be type II or type III SMA. The neuromuscular junction disorder may be myasthenia gravis or Lambert-Eaton Syndrome. The muscular dystrophy may be selected from: Duchenne muscular dystrophy, Becker muscular dystrophy, congenital muscular dystrophy, and facioscapulohumeral muscular dystrophy. The motor neuron disease may be amyotrophic lateral sclerosis. The subject may have type II or type III SMA.

Also described herein according to a third aspect there is provided a system comprising: at least one processor; and at least one non-transitory computer readable medium containing instructions that, when executed by the at least one processor, cause the at least one processor to perform a digital physiotherapy method comprising the steps of: providing a user interface to a subject that has been diagnosed as having a neuromuscular disease or disorder and optionally undergoing treatment with a therapeutic compound or composition for the treatment of the neuromuscular disease or disorder; receiving movement data comprising coordinates of a plurality of joints of the subject or data derived therefrom, at a plurality of time points, and in response to receiving movement data satisfying a set of predetermined criteria, triggering a change in the user interface, wherein the change is specific to the set of predetermined criteria satisfied and wherein the set of predetermined criteria is selected from a plurality of sets of predetermined criteria, each set identifying a predetermined movement.

The system may further comprise a display device operably coupled to the processor for display of the user interface. The system may further comprise a movement sensing device operably coupled to the process for acquisition of the movement data. The instructions may further cause the processor to perform the steps of any embodiment of the first aspect.

According to a fourth aspect, there is provided a method of improving, stabilising or reducing the degradation in a motor function score in a subject undergoing treatment with a therapeutic compound or composition for treating a neuromuscular disease or disorder, and/or increasing the likelihood of continued administration of a physiotherapy dosage regimen, wherein the method comprises implementing a computer-implemented method for digital physiotherapy comprising the steps of: providing a user interface to the subject; receiving movement data comprising coordinates of a plurality of joints of the subject or data derived therefrom, at a plurality of time points, and in response to receiving movement data satisfying a set of predetermined criteria, triggering a change in the user interface, wherein the change is specific to the set of predetermined criteria satisfied and wherein the set of predetermined criteria is selected from a plurality of sets of predetermined criteria, each set identifying a predetermined movement.

The method may have the features of any embodiment of any preceding aspect. Continued administration of a physiotherapy dosage regimen may refer to adherence to the physiotherapy dosage regimen for at least 3 months, at least 6 months, at least 9 months or at least 12 months. A predetermined physiotherapy dosage regimen may comprise the use of the computer-implemented method for digital physiotherapy for at least a predetermined amount of time and at least a predetermined frequency. The predetermined amount of time may be at least 10, 15, 20 or 25 minutes per day. The predetermined frequency may be at least 2, 3 or 4 times per week.

According to a fifth aspect, there is provided a method of treating a subject with a neuromuscular disease or disorder, wherein the method comprises the steps of: implementing a digital physiotherapy method comprising the steps of: providing a user interface to the subject; receiving movement data comprising coordinates of a plurality of joints of the subject or data derived therefrom, at a plurality of time points, and in response to receiving movement data satisfying a set of predetermined criteria, triggering a change in the user interface, wherein the change is specific to the set of predetermined criteria satisfied and wherein the set of predetermined criteria is selected from a plurality of sets of predetermined criteria, each set identifying a predetermined movement. The method may comprise administering a therapeutic compound or composition to the subject in a therapeutically effective amount. The method may improve, stabilize or reduce the degradation in a motor function score in the subject.

The method may have any one or more of the features of any preceding aspect.

The method may increase the likelihood of continued administration of a physiotherapy dosage regimen, wherein continued administration of a physiotherapy dosage regimen refers to adherence to the physiotherapy dosage regimen for at least 3 months, at least 6 months, at least 9 months or at least 12 months.

In a further aspect, the disclosure provides a system comprising at least one processor; and at least one non-transitory computer readable medium containing instructions that, when executed by the at least one processor, cause the at least one processor to perform a digital physiotherapy method comprising the steps of: providing a user interface to a subject that has been diagnosed as having a neuromuscular disease or disorder and optionally undergoing treatment with a therapeutic compound or composition for the treatment of the neuromuscular disease or disorder; receiving movement data comprising coordinates of a plurality of joints of the subject or data derived therefrom, at a plurality of time points, and in response to receiving movement data satisfying a set of predetermined criteria, triggering a change in the user interface, wherein the change is specific to the set of predetermined criteria satisfied and wherein the set of predetermined criteria is selected from a plurality of sets of predetermined criteria, each set identifying a predetermined movement.

The digital physiotherapy method may have the features of any embodiment of any preceding aspect.

In a related aspect, the disclosure provides a non-transitory computer readable medium comprising instructions that, when executed by at least one processor, cause the at least one processor to perform a digital physiotherapy method comprising the steps of: providing a user interface to a subject that has been diagnosed as having a neuromuscular disease or disorder and optionally undergoing treatment with a therapeutic compound or composition for the treatment of the neuromuscular disease or disorder; receiving movement data comprising coordinates of a plurality of joints of the subject or data derived therefrom, at a plurality of time points, and in response to receiving movement data satisfying a set of predetermined criteria, triggering a change in the user interface, wherein the change is specific to the set of predetermined criteria satisfied and wherein the set of predetermined criteria is selected from a plurality of sets of predetermined criteria, each set identifying a predetermined movement.

The digital physiotherapy method may have the features of any embodiment of any preceding aspect.

In a related aspect, the disclosure provides a computer program comprising code which, when the code is executed on a computer, causes the computer to perform a digital physiotherapy method comprising the steps of: providing a user interface to a subject that has been diagnosed as having a neuromuscular disease or disorder and optionally undergoing treatment with a therapeutic compound or composition for the treatment of the neuromuscular disease or disorder; receiving movement data comprising coordinates of a plurality of joints of the subject or data derived therefrom, at a plurality of time points, and in response to receiving movement data satisfying a set of predetermined criteria, triggering a change in the user interface, wherein the change is specific to the set of predetermined criteria satisfied and wherein the set of predetermined criteria is selected from a plurality of sets of predetermined criteria, each set identifying a predetermined movement.

The digital physiotherapy method may have the features of any embodiment of any preceding aspect.

According to a further aspect, there is provided a system comprising: a processor; and a computer readable medium comprising instructions that, when executed by the processor, cause the processor to perform the (computer-implemented) steps of the method of the first aspect. The system may further comprise one or more cameras. The one or more cameras may each be 2D or 3D cameras. A 3D camera may be a stereo camera.

According to a further aspect, there is provided a non-transitory computer readable medium or media comprising instructions that, when executed by at least one processor, cause the at least one processor to perform the method of any embodiment of any aspect described herein.

According to a further aspect, there is provided a computer program comprising code which, when the code is executed on a computer, causes the computer to perform the method of any embodiment of any aspect described herein.

### DESCRIPTION OF FIGURES

FIG. 1 shows an embodiment of a system for providing a physiotherapy to a subject, according to the disclosure.

FIG. 2 is a flow diagram showing, in schematic form, a digital physiotherapy method, according to the disclosure.

FIG. 3A illustrates schematically planes used as reference planes in embodiments of the present disclosure.

FIG. 3B illustrates schematically the location of a plurality of bodily locations (referred to herein as “joints”) for which locations may be recorded according to embodiments of the disclosure.

FIGS. 4A-4E illustrate movements (also referred to herein as “gestures”) that are used in embodiments of the present disclosure. FIG. 4A. Elbow flexion gesture. FIG. 4B. Thoracic extension gesture. FIG. 4C. Head rotation gesture. FIG. 4D. Horizontal abduction gesture. FIG. 4E. Open-close hand gesture.

FIGS. 5A-5E illustrate schematically processes for detecting the movements illustrated on FIGS. 4A-4E according to embodiments of the disclosure. FIG. 5A. Elbow flexion gesture. FIG. 5B. Thoracic extension gesture. FIG. 5C. Head rotation gesture. FIG. 5D. Horizontal abduction gesture. FIG. 5E. Open-close hand gesture.

FIG. 6 shows data for game play duration for a plurality of subjects, along days split between two cohorts: a cohort of patients with spinal muscular atrophy (SMA) and a cohort of healthy patients (neurotypical). The grey areas show minimum required playing duration between 15 and 25 min.

FIG. 7 shows the number of sessions played per participant in a feasibility study, separated between neurotypical participants (middle set of bars) and SMA participants (red set of bars).

FIGS. 8A-8B show examples of open-close gesture detection in subject 2 (SMA, FIG. 8A) and subject 1 (NT, FIG. 8B). Each figure shows angle data used to detect the gesture, the distance information between the hand tip and wrist joint, and a Boolean indicator showing detection of the gesture, for each frame (30 frames per second) of a use session by the indicated subject.

FIGS. 9A-9B show examples of elbow flexion gesture detection in subject 2 (SMA, FIG. 9A) and subject 1 (NT, FIG. 9B). Each figure shows angle data used to detect the gesture, and a Boolean indicator showing detection of the gesture, for each frame of a use session by the indicated subject.

FIGS. 10A-10B show examples of thoracic extension gesture detection in subject 2 (SMA, FIG. 10A) and subject 1 (NT, FIG. 10B). Each figure shows angle data used to detect the gesture, and a Boolean indicator showing detection of the gesture, for each frame of a use session by the indicated subject.

FIGS. 11A-11B show examples of head rotation gesture detection in subject 2 (SMA, FIG. 11A) and subject 1 (NT, FIG. 11B). Each figure shows angle data used to detect the gesture, and two Boolean indicators (left and right rotation) showing detection of the gesture, for each frame of a use session by the indicated subject.

FIGS. 12A-12B show examples of horizontal abduction gesture detection in subject 2 (SMA, FIG. 12A) and subject 1 (NT, FIG. 12B). Each figure shows angle data used to detect the gesture, and a Boolean indicator showing detection of the gesture, for each frame of a use session by the indicated subject.

### DETAILED DESCRIPTION

In describing the present invention, the following terms will be employed, and are intended to be defined as indicated below.

In this specification and the appended claims, the singular forms “a”, “an” and “the” include plural referents unless the context clearly dictates otherwise. The terms “a” (or “an”), as well as the terms “one or more,” and “at least one” can be used interchangeably herein. Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; Band C; A (alone); B (alone); and C (alone). Wherever aspects are described herein with the language “comprising,” otherwise analogous aspects described in terms of “consisting of” and/or “consisting essentially of” are also provided. The term “about” as used in connection with a numerical value throughout the specification and the claims denotes an interval of accuracy, familiar and acceptable to a person skilled in the art. In general, such interval of accuracy is ±15%. Units, prefixes, and symbols are denoted in their Système International des Unités (SI) accepted form. Numeric ranges are inclusive of the numbers defining the range. The headings provided herein are not limitations of the various aspects or aspects of the disclosure, which can be had by reference to the specification as a whole.

As used herein, the terms “computer system” of “computer device” includes the hardware, software and data storage devices for embodying a system or carrying out a computer implemented method, such as e.g. for printing a 3D object as part of a 3D printing system. For example, a computer system may comprise one or more processing units such as a central processing unit (CPU) and/or a graphical processing unit (GPU), input means, output means and data storage, which may be embodied as one or more connected computing devices. Preferably the computer system has a display or comprises a computing device that has a display to provide a visual output display (for example in the design of the business process). The data storage may comprise RAM, disk drives or other computer readable media. The computer system may include a plurality of computing devices connected by a network and able to communicate with each other over that network. For example, a computer system may be implemented as a cloud computer. The term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

The methods described herein are computer implemented unless context indicates otherwise. Indeed, the methods described herein relate to the use of a custom designed video game for the treatment of symptoms of motor neuron disease, and as such by definition requires the presence of a gaming engine, graphics processor, etc.

The methods described herein may be provided as computer programs or as computer program products or computer readable media carrying a computer program which is arranged, when run on a computer, to perform the method(s) described herein. As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

### Systems

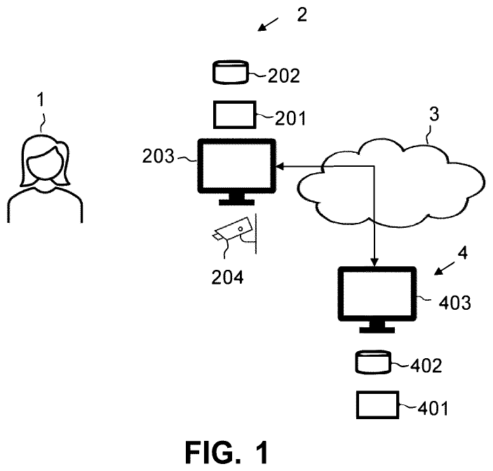

FIG. 1 shows an embodiment of a system that may be used according to embodiments of the present disclosure. For example, the system may be used for analysing motion data from a subject 1, for characterising a subject 1, for treating a subject 1, for selecting a subject 1 for participating in a clinical trial, and/or for monitoring a subject 1. The system comprises a computing device 4, which comprises a processor 401 and computer readable memory 402. In the embodiment shown, the computing device 1 also comprises a user interface 403, which is illustrated as a screen but may include any other means of conveying information to a user such as e.g. through audible or visual signals, by producing a report, etc. The computing device 4 is communicably connected, such as e.g. through a network 3, to a digital physiotherapy system 2. In the illustrated embodiment the digital physiotherapy system comprising a display device 203, a processor 201, a memory 202 and one or more motion sensors 204, here illustrated as a camera. The display device is used to display a gaming interface to the subject 1. The one or more motion sensors (e.g. cameras) 204 are used to record movements of the subject 1, and the recorded movements are analysed by the processor 201 or the processor 401 using instructions stored on memories 402 and/or 202. The analysis produces instructions to a gaming engine executed by the processor 201 or the processor 401. The gaming engine may execute instructions stored on memories 402 and/or 202. In response to receiving instructions generated by processors 401 and/or 201, the gaming engine controls the display of an interface on the display 203, thereby enabling the subject 1 to interact with said interface through their movements. The computing device 4 may be a server, smartphone, tablet, personal computer or other computing device. The computing device is configured to implement methods as described herein. For example, the digital physiotherapy system 2 may be configured to communicate with the remote computing device 4, to implement a method of delivering digital physiotherapy or any other method described herein. In alternative embodiments, the digital physiotherapy system 2 may be configured to communicate with the remote computing device 4 to obtain instructions that can be used by the digital physiotherapy system 2 to implement a method of delivering digital physiotherapy or any other method described herein. In such embodiments, the digital physiotherapy system may also be configured to send the result of the method to the remote computing device 4. Communication between the digital physiotherapy system 2 and the remote computing device 4 may be through a wired or wireless connection, and may occur over a local or public network 3 such as e.g. over the public internet. The motion sensors 204 and display 203 are typically in wired connection with the processor 201, but they may instead or in addition be in wired or wireless connection with computing device 4 and/or in wireless connection with processor 201. Any of the steps of any method described herein may be implemented by processor 201 and/or processor 401, executing instructions stored on memory 202 and/or memory 402. The display 203 may be a TV monitor, a screen or a laptop. The subject may be located with their chest approximately 150 cm from the camera 204. The subject may be located between 150 cm and 300 cm from the display 203. The subject may be sat down e.g. on a chair, seat or wheelchair. The motion sensor 204 may be in wired connection with the screen 203, and the screen may be associated with processor 201 in wireless connection (such as e.g. through the internet) with computing device 4. Computing device 4 may be a server. Computing device 4 may not comprise a user interface 403. Computing device 4 may be configured to store data (such as e.g. any output of any step of any method described herein, and movement data, any gaming data, etc.) on memory 402 and/or to provide such data to a further computing device (not shown) such as e.g. a computing device associated with a healthcare professional.

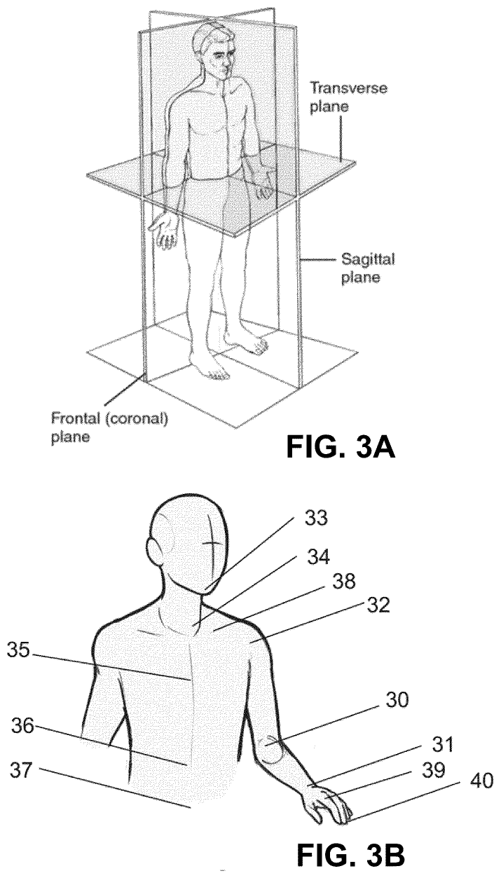

As used herein, the term “motion sensor” refers to a sensor that is capable of measuring the position of one or more features of a subject (also referred to herein as “joints”). The position of the one or more features may be measured in 3D or in 2D. Positions measured in 2D may be measured in a plurality of different 2D planes (such as e.g. a plurality of the sagittal, frontal or transverse planes of a subject). Thus, the position of a feature of a subject may comprise a 3D position and/or one or more 2D positions. The motion sensor is preferably a markerless motion sensor. A markerless motion sensor is a sensor that does not rely on the presence of a specific marker (e.g. reflective marker) on the subject. The motion sensor may be configured to acquire measurement in a continuous manner. As the skilled person understands, continuous measurement refers to the acquisition of measurements at a predetermined sampling frequency. In other words, continuous measurements refer to the acquisition of time series comprising data at each of a plurality of time frames. The time frames are defined by the sampling frequency of the sensor. The predetermined sampling frequency may also be referred to as the nominal sampling frequency of the sensor. For example, a sensor may have a nominal sampling frequency is 30 Hz, which should result in measurements recorded every 33 ms. In practice the sampling rate of a sensor may not be perfectly consistent, and some variation around the nominal sampling frequency may be expected. For example, using a sensor with a nominal sampling frequency of 30 Hz may result in time series comprising time frames that are usually separated by 33 ms, as well as time frames separated by longer periods such as e.g. 100 ms, or shorter periods. In embodiments, a sensor with a nominal sampling rate of 30 Hz may have an effective sampling rate of at least 25 Hz. The effective sampling rate is the sampling rate that is actually achieved by the sensor. The nominal sampling rate is the sampling rate that the sensor aims to achieve, such as for example on average. A sensor may have a sampling rate that is variable around the nominal sampling rate, but where at all times the sampling rate (effective sampling rate) is above a predetermined threshold. Preferably the sensor used in the methods of the present invention has an effective sampling rate of at least 10 Hz. The motion sensor may comprise one or more cameras. A camera may be a 2D camera or a 3D camera. A 2D camera may be for example a Webcam connected to a computing device. A 2D camera may be a device configured to obtain 2 dimensional images of a subject. A plurality of 2D images captured at a predetermined sampling rate (e.g. 30 images per second) may be used to simulate a movement. For example, such a plurality of images can be analysed through known image analysis processes to extract information that is indicative of the movements that were performed while the data was acquired. In embodiments, the information may comprise 3D coordinates of one or more features of a subject. In embodiments, a motion sensor comprising a plurality of 2D cameras positioned at different angles relative to a subject may be configured to measure the 2D position of one or more features of the subject and derive 3D coordinates from these. In embodiments, the information may comprise one or more motion metrics for one or more features of a subject. The one or more motion metrics may comprise for example displacement of a feature of the subject, angles between vectors derived from features of the subject (vectors between specific joints), angles between a vector derived from features of the subject (e.g. a vector between specific joints) and a predetermined vector or direction (e.g. direction perpendicular to the ground), combinations of the above and versions of the above where the displacement/angle is expressed in a particular plane (e.g. any of the sagittal, frontal or transverse plane) of the subject corresponding to the angle of view of the 2D camera. For example, software such as kinovea (www.kinovea.org) provide functionalities to automatically measure angles and distances and follow trajectories of points on a video. Each of these metrics may provide an indication of a movement that is being performed, which can be used in the methods of the present disclosure. 2D cameras are advantageously cheap and widely available. A camera may be a 3D camera, such as a stereo camera. A 3D camera may be a device configured to obtain information comprising 3D images. A 3D camera may be used to obtain 3D position information for one or more features of a subject, for example derived from information comprising the 2D coordinates (e.g. x, y coordinates) and depth (e.g. z coordinate) of one or more features of a subject. The features for which a position may be measured may comprise one or more joints of a subject, as further described below. A motion sensor comprising a 3D camera may be selected from: Microsoft Kinect, Intel Realsense, Orbbec, and OAK-D. The Microsoft Kinect motion sensor (more information at: azure.microsoft.com/en-us/services/kinect-dk/) may be able to measure the 3D coordinates of 32 joints. The Intel Realsense sensor (more information at: www.intelrealsense.com) may be able to measure the 3D coordinates of 18 joints. The Orbbec sensor (more information at: orbbec3d.com) may be able to measure the 3D coordinates of 19 joints. In embodiments, the motion sensor is a Microsoft Kinect sensor. The present inventors found this sensor to provide very high accuracy and detailed information about a subject's movements. Any movement sensor that can record the 3D localisation (e.g. coordinates in a 3D reference system) of a plurality of joints in real time (i.e. at a sampling rate of at least 10 Hz, such as e.g. between 10 and 40 Hz, where a minimum sampling rate may refer to an effective sampling rate), such as illustrated on FIG. 3B, may be used in the context of the present disclosure. In embodiments, the movement sensor is configured to record the localisation of at least the head, neck, chest, shoulders, elbows, wrists, hands, and hand-tips of a subject.

### Digital Physiotherapy Methods and Tools

The present disclosure provides methods of treating neuromuscular diseases using a therapeutic in combination with a digital physiotherapy tool that enables a user to perform physiotherapy exercises by interacting with a user interface.

In Chen et al. 2017, the present inventors described a game based on the Microsoft Kinect sensor, specifically designed to measure active upper limb movement. The game was tested in ambulant SMA type III patients, where the three-dimensional coordinates of 9 upper body points (head, neck, shoulders, elbows, hands and torso) was recorded over the course of the game and used to derive movement features such as the extension and flexion angles of the elbows and the lifting angles of the arms, the speed of motion and the reachable spatial projection into a two-dimensional plane (maximal area of the 2D plane reached by the hands during the task). The game was used as a test for upper limb movement and lasted less than 5 minutes. The game comprised a plurality of predetermined tasks (20 in total), each to be performed within 12 seconds, and each comprising the subject having to reach (elbow extension) to cause an avatar to take an object (an item of clothing) on a shelf displayed on a user interface, then flex the elbow to cause the avatar to place the object on itself. The game was not designed or studied as a physiotherapy tool, and only comprised a single type of exercise (single movement). None of the movement features tested showed any difference in SMA type III patients compared to healthy controls. By contrast, the methods described herein may enable the calculation of different metrics, which may discriminate between subjects with neuromuscular disorders (particularly subjects with SMA type II) and healthy subjects, as well as be indicative of motor function, for example predicting clinical metrics of motor function.

Blaschek et al. (2022) described the use of a markerless motion tracking method using an RGB-depth sensor to quantify the Children's Hospital of Philadelphia Infant Test of Neuromuscular Disorders (CHOP INTEND) scores, by recording the subjects (2-46 months old) for 2 minutes during unperturbed spontaneous whole-body activity. Lowes et al. (2015) described a tool based on the Microsoft Kinect sensor, for determining the furthest arm excursion in all planes in subjects with dystrophinopathy, from which a total reachable area scaled by arm length was obtained. This was shown to discriminate between subjects with dystrophinopathy and controls. These studies demonstrate the feasibility of the use of a Kinect based interface in subjects with neuromuscular disorders, but do not investigate the use of such technology as part of a digital physiotherapy tool.

The term “digital physiotherapy” tool may refer to a computing device configured to implement a digital physiotherapy method as described herein, to a computer program comprising code that when executed on a computer causes the computer to implement a digital physiotherapy method as described herein, or a tangible computer readable medium storing instructions that when executed by a processor cause the processor to implement a digital physiotherapy method as described herein (also referred to as “exergame”). The terms “digital physiotherapy” may refer to the computing device, computer program product, computer readable medium or computer implemented method. A digital physiotherapy method is a computer implemented method comprising steps as described herein. The term “digital physiotherapy device” refers to a computing device configured to implement a digital physiotherapy method as described herein, or to a system comprising such a computing device and one or more cameras. The computing device may be a user device (such as e.g. a laptop, phone, tablet, personal computer, etc.). The computing device may locally store and execute instructions to implement one or more steps of a digital physiotherapy method as described herein. The computing device may communicate with a remote computer configured to implement one or more steps of a digital physiotherapy method as described herein, such as e.g. through a public or private network, as described above.

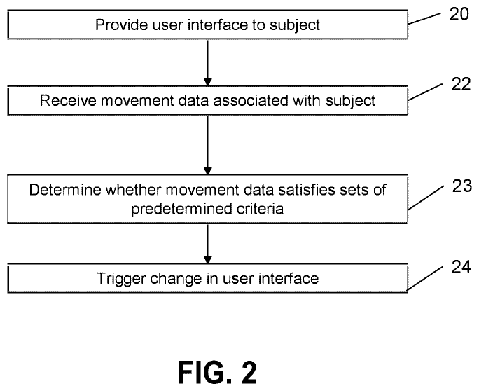

FIG. 2 is a flow diagram showing, in schematic form, a digital physiotherapy method, according to the disclosure. A digital physiotherapy method may comprise: at step 20, providing a user interface to a subject that has been diagnosed as having a neuromuscular disease; at step 22, receiving movement data comprising coordinates of a plurality of joints of a subject or data derived therefrom, at a plurality of time points, and in response to receiving movement data satisfying a set of predetermined criteria, triggering a change in the user interface at step 24, wherein the change is specific to the set of predetermined criteria satisfied. The predetermined criteria may comprise a plurality of sets of predetermined criteria that each identify a predetermined movement. Thus, movement data satisfying a first set of predetermined criteria may be identified as indicative of the subject having performed a first predetermined movement, movement data satisfying a second set of predetermined criteria may be identified as indicative of the subject having performed a second predetermined movement, etc. Each set of predetermined criteria may comprise one or more criteria. Each set of predetermined criteria may be associated with a corresponding predetermined label identifying the predetermined movement. Each criterion may apply to a movement data for a predetermined joint or set of joints, or data derived therefrom. A predetermined movement may be selected from: an elbow flexion gesture, a thoracic extension gesture, a head rotation gesture, a horizontal abduction gesture, and an open-close hand gesture. The method may comprise step 23 of determining whether the movement data satisfies each of one or more sets of predetermined criteria. Movement data that satisfies a particular set of predetermined criteria may be associated with a corresponding predetermined label.

The method may comprise repeating steps 20 to 23 for a plurality of iterations, wherein the set of predetermined criteria or plurality of sets of predetermined criteria are selected independently for each iteration. Thus, the set of predetermined criteria or plurality of predetermined criteria may be different between respective iterations of the methods. The set of predetermined criteria or plurality of sets of predetermined criteria may be selected from a common set. For example, at a first iteration of the method, step 24 may be performed in response to receiving movement data satisfying a first set of predetermined criteria corresponding to a first set of one or more predetermined movements. At a second iteration of the method, step 24 may be performed in response to receiving movement data satisfying a second set of predetermined criteria corresponding to a second set of one or more predetermined movements, where the second set may be different from the first set. Thus, the methods described herein may prompt a subject to perform one or more movements at a first time point and one or more movements that may be the same or different at a second time point. At each iteration, a set of predetermined criteria that triggers a change in the user interface may be selected from a plurality of sets of predetermined criteria. The selection may be random, the selection may be random with uniform or non-uniform probability. The selection may be according to one or more predetermined scenarios (e.g. if user interface shows x, then only movements y and z are possible, and trigger changes v and 2, respectively). The user interface that is displayed at step 20 may provide information indicative of the set of predetermined criteria that would trigger a change in the user interface. This information may comprise information identifying one or more movements to be performed, said movements being associated with the set of predetermined criteria that would trigger a change in the user interface. Instead or in addition to this, the information may comprise information that a user may be able to associate with predetermined movements based on a set of rules.

The set of predetermined criteria that apply at any one or more iterations of a method as described herein may be defined for example by a healthcare practitioner, such as e.g. a physiotherapist. The set of predetermined criteria that apply at any one or more iterations of a method may be set prior to and/or may be changed during a course of treatment/use of the digital physiotherapy tool by a subject, such as e.g. to reflect the subject's changing abilities and/or physiotherapy objectives set by a healthcare practitioner. As a specific example, a first set of predetermined criteria may be set at the start of a course of treatment/use of the digital physiotherapy tool (e.g. at one or more iterations of the methods that are implemented at the start or a course of treatment/use of the tool by a subject) which comprises one or more movements. After an amount of time of treatment with/use of the digital physiotherapy tool or a number of iterations of the method (i.e. after the subject has successfully performed the predetermined movements for each iteration), a second set of predetermined criteria may be set. This may ensure that the subject keeps being challenged to perform different movements as their motor abilities change. As another example, a different set of predetermined criteria may be set for a first subject than for a second subject. This may ensure that both subjects are performing movements that are adapted to their motor abilities and physiotherapy objectives.

Providing a user interface to a subject may comprise causing a user interface to be displayed on a display. Triggering a change in the user interface may comprise causing a user interface displayed on a display to show different information.

Receiving movement data comprising coordinates of a plurality of joints of the subject or data derived therefrom at a plurality of time points may comprise receiving data from one or more cameras (directly or indirectly, such as e.g. through a computing device associated with the one or more cameras).

The term “joint” as used herein refers to a location on the body of a subject, the spatial position of which can be measured using a motion sensor as described herein. A joint is typically but not necessarily located on a physical joint of the subject. A joint may be selected from a location that aligns with a subject's: left elbow, right elbow, left wrist, right wrist, left shoulder, right shoulder, head (which may be a single location or a location selected from or derived from a plurality of locations, such as e.g. the location of a right ear, the location of a left ear, the location of a left eye, the location of a right eye, and the location of the nose), neck, spine at chest level, spine at navel level, pelvis, left clavicle, right clavicle, left hand, right hand, left hand tip, and right hand tip. In embodiments, the methods described use one or more, preferably all, of the following joints: head, neck, spine at chest level, left shoulder, right shoulder, left elbow, right elbow, left wrist, right wrist, left hand, right hand, left hand-tip, and right hand-tip.

Depending on the motion sensor used, a joint may be associated with one or more of: one or more sets of 2D coordinates (e.g. a set of 2D coordinates for each of a plurality of 2D cameras), a set of 3D coordinates (e.g. a set of 3D coordinates measured by a 3D camera or derived from 2D coordinates from one or more 3D cameras), and one or more orientation vectors. An orientation vector may be a 3D unit length vector that originates at the location of a joint and indicates one direction (which may be one of 3 directions of a 3D orthogonal coordinate system located with the joint at its origin). The direction (location) of an orientation vector (or an orthogonal basis comprising such a vector) may be defined for a default orientation of the joint, such that the measured location of the orientation vector provides information about the orientation of the joint relative to its default orientation. For example, a head joint may be defined to be associated by default with a forward orientation vector that points directly towards the sensor when the subject's head is straight and pointing towards the sensor. When the subject's head is rotated the forward orientation vector will rotate towards the left or right compared to this default position. This rotation can be identified based on the detected location of the forward vector. A joint may be associated with a forward orientation vector and a downward and/or upward orientation vector. The forward and downward and/or upward orientation vectors may be unit length vectors. The forward and downward and/or upward orientation vectors may be orthogonal. Downward and upward orientation vectors may be collinear. Downward and upward orientation vectors may be vectors that point to opposite directions along the same axis. Downward and upward orientation vectors may be vectors that point to opposite directions along the same axis. Thus, it may be possible to determine a downward vector from an upward vector and vice-versa, such that only one of the downward vector and upward vector for a joint may be provided.

Predetermined movements may also be referred to herein as “gestures”. The predetermined movements may be selected from: elbow flexion, horizontal abduction, head rotation, thoracic extension and open-close hand. A predetermined movement may be a single gesture or a sequential gesture. A single gesture may be associated with a single predetermined criterion. Single gestures may be detected by evaluating the corresponding predetermined criterion, and when the predetermined criterion is met, determining that the corresponding single gesture has been performed. A sequential gesture may be associated with a plurality of predetermined criteria. Movement data may be considered to satisfy the plurality of criteria when the movement data comprises movement data at a first time point that satisfies a first criterion of the plurality of criteria, movement data at a second (subsequent) time point that satisfies a second criterion of the plurality of criteria, etc. A sequential gesture may be associated with a first predetermined criterion and at least a second predetermined criterion. In other words, a sequential gesture may be associated with a sequence of predetermined criteria. Movement data may be indicative of a sequential gesture when it comprises movement data at a first time point that satisfies the first predetermined criterion and movement data at a second time point that satisfies the second predetermined criterion (and optionally movement data at a third, fourth, etc. time point(s) that satisfy respective predetermined criteria, depending on the number of criteria in the sequential gesture). Sequential gestures may be detected by evaluating the first predetermined criterion, and when the first predetermined criterion is met, evaluating the second predetermined criterion (and so on if the sequential gesture is associated with more than two predetermined criteria), and when all predetermined criteria associated with the sequential gesture have been sequentially met, determining that the sequential gesture has been performed. Sequential gestures may be gestures that are associated with a plurality of phases of movement, such as e.g. lifting then lowering an arm, rotating the head to one side then back to the front, opening then closing hand (and more complex versions of these such as opening-closing-opening the hand, rotating the head to one side, to the front then to the other side, etc.) Sequential gestures may be selected such that the second (and subsequent) criteria of a sequential gesture will necessarily become true eventually (e.g. with a head rotation to the left the subject will always eventually turn the head back to face the sensor). This enables the method to look for fulfillment of the second criterion as soon as the first criterion is fulfilled without imposing any speed of movement requirement (timeframe for search of movement data satisfying the second criterion). Examples of single gestures may include an elbow flexion and a horizontal abduction. Examples of sequential gestures may include a head rotation, a thoracic extension and an open-close hand gesture. All gestures may be detected in “real time”. In other words, movement data received from a movement sensor may be evaluated as it is received to determine whether the movement data satisfies any of a plurality of predetermined criteria associated with corresponding gestures. Every time new data (e.g. a new frame) is obtained, the movement data may be evaluated to determine whether any of the predetermined criteria is satisfied in view of the new data received. When a predetermined criterion associated with a single gesture is satisfied, the method may comprise determining that the gesture has been performed. Any subsequently received data may be evaluated for the plurality of predetermined criteria associated with corresponding gestures. Optionally, an action associated with performance of the gesture may be implemented, such as e.g. causing a change in a user interface. When a predetermined criterion associated with a sequential gesture is satisfied, the method may comprise evaluating subsequently received movement data by determining whether a subsequent predetermined criterion associated with the sequential gesture is satisfied. When the last received and evaluated data received meets the subsequent predetermined criterion associated with the sequential gesture, the method may comprise determining that the sequential gesture has been performed (when the subsequent predetermined criterion was the last criterion for the sequential gesture), or evaluating subsequently received movement data by determining whether a further subsequent predetermined criterion associated with the sequential gesture is satisfied. When the last received and evaluated data received meets the last subsequent predetermined criterion associated with the sequential gesture, the method may comprise determining that the sequential gesture has been performed. Optionally, an action associated with performance of the gesture may be implemented, such as e.g. causing a change in a user interface.

Any predetermined criterion may individually apply to angles between predetermined vectors derived from one or more joints, or to projections of such angles on predetermined body planes. Conventional body planes that can be used as predetermined body planes are illustrated on FIG. 3A and include a transverse plane, a sagittal plane, and a frontal (also referred to as “coronal) plane. The transverse plane as illustrated on FIG. 3A is a plane that is perpendicular to the main axis of the subject's body. The sagittal plane as illustrated on FIG. 3A is a plane that separates the body between a left-hand side and a right-hand side. The frontal plane as illustrated on FIG. 3A is a plane that separates the body between a front side and a back side. Predetermined criteria may apply to angles between predetermined vectors derived from one or more joints, and/or to projections of such angles on the transverse plane. Predetermined vectors derived from one or more joints may be selected from: a forearm vector, an upper arm vector, a chest vector, a head forward vector, a torso forward vector, an arm vector, a torso down vector, a torso up vector, a finger vector, and a palm vector. All vectors may be 3D vectors defined by two sets of 3D coordinates, or 3D vectors defined by multiple 3D vectors each defined by two sets of 3D coordinates. Each set of 3D coordinates may correspond to the 3D coordinates of a particular joint, or 3D coordinates derived from a plurality of joints (such as e.g. by averaging or other summary metric). A forearm vector may be a vector that connects the 3D location of an elbow joint and the 3D location of any point located between the wrist and hand tip of the subject on the same side (e.g. left arm or right arm), such as e.g. a wrist joint, hand joint or hand tip joint. In embodiments, a forearm vector is a vector that connects the 3D locations of an elbow joint and a wrist joint on the same side. An upper arm vector may be a vector that connects the 3D location of an elbow joint and the 3D location of any point located between the shoulder and neck of the subject on the same side (e.g. left arm or right arm), such as e.g. a shoulder joint, clavicle joint or neck joint. In embodiments, an upper arm vector is a vector that connects the 3D locations of an elbow joint and a shoulder joint on the same side. A chest vector may be a vector that is the average of a vector that connects any two points between the shoulder and neck of the subject on the same side (such as e.g. a vector that connects the clavicle and shoulder joints of the subject on the same side), and a vector that connects the neck and elbow joints of the subject on the same side. A head forward vector may be a vector that connects the head joint of a subject and a point forward of the head joint (i.e. towards the movement sensor) at the same height. The forward direction may be defined by an orientation vector associated with the head joint. A torso forward vector may be a vector that is the average of a plurality of vectors that each connect a joint on the torso of the subject and a point forward of the respective joint (i.e. towards the movement sensor) at the same height. The forward direction of such a torso vector may be defined using a respective orientation (forward vector) associated with each of the neck, spine chest, spine naval and the pelvis joint (such as e.g. by obtaining the average of these orientation vectors). The joints on the torso may be aligned with the central axis of the body (i.e. around the sagittal plane, or aligned with the spine). The joints on the torso may be located anywhere between the neck and pelvis of the subject. For example, the joints on the chest may be selected from: the neck, chest (e.g. breast level), navel or pelvis. In embodiments, the torso forward vector may be a vector that is the average of a plurality of vectors that each connect a joint of a plurality of joints on the torso of the subject and a point forward or the respective joint at the same height, wherein the plurality of joints on the torso of the subject comprise any combination of: a neck joint, a chest joint, a navel joint and/or a pelvis joint, or all of the above. The point(s) forward of any joint may be defined such that the resulting vector(s) has/have unit length. In other words, vectors such as head forward vectors and torso forward vectors may have unit length by convention, and their direction may be derived from one or more orientations vectors associated with the respective one or more joints from which these vectors originate. A wrist to shoulder vector may be a vector that connects the 3D location of any point located between the wrist and hand tip of the subject and the 3D location of any point located between the shoulder and neck of the subject on the same side (e.g. left arm or right arm), such as e.g. a vector that connects any of a wrist joint, hand joint or hand tip joint to any of a shoulder joint, clavicle joint or neck joint on the same side. In embodiments, a wrist to shoulder vector connects the 3D location of a wrist joint and the 3D location of a shoulder joint. An arm vector may be a vector that is the average of a plurality of vectors that each connect a pair of joints along an arm of the subject (i.e. joints on the same side). For example, an arm vector may be the average of an upper arm vector, a forearm vector and a wrist to shoulder vector. The present inventors have identified that such an arm vector can be located with higher accuracy than e.g. just using the wrist to shoulder vector, at least with some movement sensors, which was found to lead to more accurate detection of gestures involving arm movement. A torso down vector may be a vector that is the average of a plurality of vectors that each connect a joint on the torso of the subject and a point downward of the respective joint. The downward direction may be defined using a respective orientation (downward vector) associated with each of the neck, spine chest, spine naval and the pelvis joint (such as e.g. by obtaining the average of these orientation vectors). The point(s) downward of any joint may be defined such that the resulting vector(s) has/have unit length. In other words, vectors such as the torso down vector may have unit length by convention, and their direction may be derived from one or more orientations vectors associated with the respective one or more joints from which these vectors originate. The joints on the torso may be aligned with the central axis of the body (i.e. around the sagittal plane, or aligned with the spine). The joints on the torso may be located anywhere between the neck and pelvis of the subject. For example, the joints on the chest may be selected from: the neck, chest (e.g. breast level), navel or pelvis. In embodiments, the torso down vector may be a vector that is the average of a plurality of vectors that each connect a joint of a plurality of joints on the torso of the subject and a point downward or the respective joint, wherein the plurality of joints on the torso of the subject comprise any combination of: a neck joint, a chest joint, a navel joint and/or a pelvis joint, or all of the above. A torso up vector may be a vector that is the average of a plurality of vectors that each connect a joint on the torso of the subject and a point upward of the respective joint. The upward direction may be defined using a respective orientation (upward vector) associated with each of the neck, spine chest, spine naval and the pelvis joint (such as e.g. by obtaining the average of these orientation vectors). The point(s) upward of any joint may be defined such that the resulting vector(s) has/have unit length. In other words, vectors such as the torso up vector may have unit length by convention, and their direction may be derived from one or more orientations vectors associated with the respective one or more joints from which these vectors originate. The joints on the torso may be aligned with the central axis of the body (i.e. around the sagittal plane, or aligned with the spine). The joints on the torso may be located anywhere between the neck and pelvis of the subject. For example, the joints on the chest may be selected from: the neck, chest (e.g. breast level), navel or pelvis. In embodiments, the torso up vector may be a vector that is the average of a plurality of vectors that each connect a joint of a plurality of joints on the torso of the subject and a point upward or the respective joint, wherein the plurality of joints on the torso of the subject comprise any combination of: a neck joint, a chest joint, a navel joint and/or a pelvis joint, or all of the above. The present inventors have identified that the use of a plurality of joints for determining a torso orientation vector (such as e.g. the torso forward vector, the torso up vector and the torso down vector), for example by obtaining the average of forward/down/up orientation vectors associated with the plurality of joints, resulted in more accurate detection of torso movements than using a single joint located on the torso. A finger vector may be a vector that connects the 3D location of any point between the wrist and hand (anywhere on the palm) of the subject and the 3D location of any point located between the hand (anywhere on the palm) and the fingertips of the subject on the same side (e.g. left arm or right arm). In embodiments, a finger vector is a vector that connects the 3D locations of a hand joint and a hand tip joint on the same side. A palm vector may be a vector that connects the 3D location of the wrist of the subject and the 3D location of the hand (anywhere on the palm) of the subject on the same side (e.g. left arm or right arm).

Any predetermined criterion as described herein may be subject to calibration. Calibration may be performed or may have been performed by recording movement data of the subject while the subject is performing a known gesture. For any criterion to be calibrated, a predetermined angle that is the subject of the criterion may be quantified while the subject is performing a known gesture to which the criterion pertains. A range of angles that includes the one or more angles quantified may be selected as the range of angles applicable according to the predetermined criterion. In other words, when determining whether movement data satisfies the predetermined criterion, angles that fall within the range of angles identified during calibration may be considered to satisfy the predetermined criterion. Thus, any methods described herein may comprise a step of obtaining movement data associated with the subject while the subject is performing a known gesture, and identifying one or more predetermined criteria associated with the known gesture as one or more ranges for respective predetermined angles observed in the movement data. The methods may further comprise prompting the subject to perform the known gesture, and recording movement data associated with the subject while they are performing the known gesture. For example, a character may be displayed on the user interface to show the subject the gesture to be performed. The subject may be prompted to adopt the particular posture (e.g. that adopted by the character displayed on screen). The positions of the relevant joints, derived vectors and/or angles between derived vectors may be recorded while the subject is holding the posture. The calibration process may be performed for one or more predetermined gestures, such as e.g. one or more or all of the gestures selected from: elbow flexion, horizontal abduction, head rotation, thoracic extension and open-close hand. A calibration process may be performed at a first time point, such as e.g. prior to performing a first instance of steps 22-24 as described in relation to FIG. 2. The calibration process may be performed at a further time point, such as e.g. prior to performing a further instance of steps 22-24 as described in relation to FIG. 2. The further time point may be separated from the first time point by a predetermined amount of time. The predetermined amount of time may be defined in absolute terms or in terms of amount of active or total time of use of the digital physiotherapy tool. The further time point may be separated from the first time point by any arbitrary amount of time. For example, the calibration process may be repeated at any point on receiving of a request from the subject (or any other user) to perform a calibration process. Any predetermined criterion as described herein may be used with default values in the absence of calibrated values. The calibration process may advantageously adapt the predetermined criteria for gesture detection to a subject's particular physical abilities. Calibration may be run automatically the first time that the digital physiotherapy tool is launched.

A calibration process may be subject to a range of possible values for each of one or more predetermined criteria. In other words, calibration may be within a predetermined range, such that calibrated values for a predetermined criterion must fall within the predetermined range. Movement data that does not fall within the predetermined range may not be considered to meet the predetermined criterion for the gesture to be detected during calibration and/or may not be considered suitable for calibration. In embodiments, when movement data recorded during calibration of a predetermined criterion does not fall within a predetermined range for said predetermined criterion, the calibrated value may be set to the nearest value that is within the predetermined range. The predetermined range for calibration may be defined for example by a healthcare practitioner, such as e.g. a physiotherapist. The predetermined range for calibration may be set prior to and/or may be changed during a course of treatment/use of the digital physiotherapy tool by a subject, such as e.g. to reflect the subject's changing abilities and/or physiotherapy objectives set by a healthcare practitioner. Similarly, any predetermined criterion associated with a gesture may be set prior to and/or changed during a course of treatment/use of the digital physiotherapy tool by a subject, such as e.g. to reflect the subject's changing abilities and/or physiotherapy objectives set by a healthcare practitioner. In other words, the digital physiotherapy tool can be configured such that the subject must perform gestures that satisfy predetermined criteria (optionally with some tolerance set by calibration), which criteria may be set and/or modified during use to correspond to physiotherapy objectives set by a healthcare practitioner. As a specific example, a predetermined criterion may be set to a lower value (lower range of movement) or a wider and/or lower predetermined range for calibration at the start of a course of treatment/use of the digital physiotherapy tool than after an amount of time of treatment with/use of the digital physiotherapy tool. This may ensure that the subject keeps being challenged to perform a wider range of movements as their motor abilities improve. As another example, a predetermined criterion may be set to a lower value (lower range of movement) or a wider and/or lower predetermined range for calibration for a first subject than for a second subject. This may ensure that both subjects are performing movements within a range that is adapted to their motor abilities.

An elbow flexion may be associated with a single predetermined criterion. The single predetermined criterion may be that the angle between a forearm vector and an upper arm vector on the same side (i.e. left forearm vector and left upper arm vector, or right forearm vector and right upper arm vector) changes between a first predetermined value and a second predetermined value. The first and/or second predetermined values may be any values identified through a calibration process as described herein. In embodiments, the first predetermined value may be set to a fixed default value. In embodiments, the second predetermined value is identified through calibration (or set to a default value in the absence of calibration). The first predetermined value may be about 110 degrees, about 100 degrees, about 90 degrees, or between 90 and 110 degrees. The second predetermined values may be about 20 degrees, about 30 degrees, about 40 degrees, about 50 degrees, or between 20 and 50 degrees. For example, an elbow flexion may be associated with an angle between a forearm vector and an upper arm vector changing from a value between 90 degrees and 110 degrees (e.g. 100 degrees) to a value between 20 degrees and 50 degrees (e.g. 30 degrees). In other words, an elbow flexion may be a gesture that starts with movement data in which the angle between a forearm vector and an upper arm vector is between 90 degrees and 110 degrees (e.g. 100 degrees) and finishes with movement data in which this angle is between 20 degrees and 50 degrees (e.g. 30 degrees). Alternatively, the single predetermined criterion may be that the angle between a forearm vector and an upper arm vector on the same side (i.e. left forearm vector and left upper arm vector, or right forearm vector and right upper arm vector) is below a predetermined value. The predetermined value may be a value within a predetermined range, subject to calibration. The predetermined value may be between 30 and 100 degrees. The predetermined value may be in a range of 65±35 degrees.

A thoracic extension may be associated with a plurality of predetermined criteria that apply to respective first and subsequent movement data (i.e. criteria that apply to movement data at different time points). A first predetermined criterion may be that the angle between the left and right chest vectors is within a first predetermined range, or is above a predetermined threshold value that is within the first predetermined range. The first predetermined range may be any range identified through a calibration process as described herein. The first predetermined range may be 190±12 degrees. A second predetermined criterion may be that the angle between the left and right chest vectors is within a second predetermined range. The second predetermined range may be any range identified through a calibration process as described herein. The second predetermined range may be 160±15 degrees. The second predetermined criterion may apply to movement data that is subsequent in time to the movement data that satisfies the first predetermined criterion. In other words, meeting the second predetermined criterion may be conditional on the first predetermined criterion having been met. Thus, a thoracic extension may be identified in movement data where the movement data at a first time point satisfies the first predetermined criterion and movement data at a second time point subsequent to the first time point meets the second predetermined criterion.

A head rotation may be associated with a plurality of predetermined criteria that apply to respective first and subsequent movement data (i.e. criteria that apply to movement data at different time points). A first predetermined criterion may be that the angle between the head forward vector and the torso forward vector is within a first predetermined range, or above a first predetermined threshold that is in said first predetermined range. The first predetermined range may be any range identified through a calibration process as described herein. The first predetermined range may be 42±5 degrees (towards either side, i.e. left or right, of the sagittal plane). A second predetermined criterion may be that the angle between the head forward vector and the torso forward vector is within a second predetermined range, or below a second predetermined threshold that is within said second predetermined range. The second predetermined range may be any range identified through a calibration process as described herein. The second predetermined range may be 0±10 degrees (i.e. within a predetermined range around the forward direction/sagittal plane). The second predetermined criterion may apply to movement data that is subsequent in time to the movement data that satisfies the first predetermined criterion. In other words, meeting the second predetermined criterion may be conditional on the first predetermined criterion having been met. Thus, a head rotation may be identified in movement data where the movement data at a first time point satisfies the first predetermined criterion and movement data at a second time point subsequent to the first time point meets the second predetermined criterion.

A horizontal abduction may be associated with a plurality of predetermined criteria that apply to movement data at the same time point. A horizontal abduction may be associated with a first predetermined criterion that applies to the angle between the torso forward vector and an arm vector (on the left or right side). A first predetermined criterion may be that the angle between the torso forward vector and an arm vector is within a first predetermined range, or below a first predetermined threshold value that is within said first predetermined range. The first predetermined range may be any range identified through a calibration process as described herein. The first predetermined range may be between 0 and 70 degrees. Note that in practice the first predetermined range may be expressed in negative values depending on the direction of movement of the arm (i.e. the range may be expressed as between 0 and −70 degrees on one side, and between 0 and 70 degrees on the other side). A horizontal abduction may be associated with a second predetermined criterion that applies to the angle between the torso down vector and the upper arm vector (on the same side as that to which the first predetermined criterion applies). A second predetermined criterion may be that the angle between the torso down vector and the upper arm vector is within a second predetermined range, or above a second predetermined threshold value that is within said second predetermined range. The second predetermined range may be any range identified through a calibration process as described herein. The second predetermined range may be between 30 and 80 degrees. A horizontal abduction may be associated with a third predetermined criterion that applies to the angle between the torso up vector and the forearm arm vector (on the same side as that to which the first predetermined criterion applies). A third predetermined criterion may be that the angle between the torso up vector and the forearm vector is within a third predetermined range, or above a third predetermined threshold value that is within said third predetermined range. The third predetermined criterion may ensure that the forearm is approximately perpendicular to the body when performing the gesture. The third predetermined range may be any range identified through a calibration process as described herein. The third predetermined range may be 95±18 degrees (i.e. between 77 and 113 degrees).

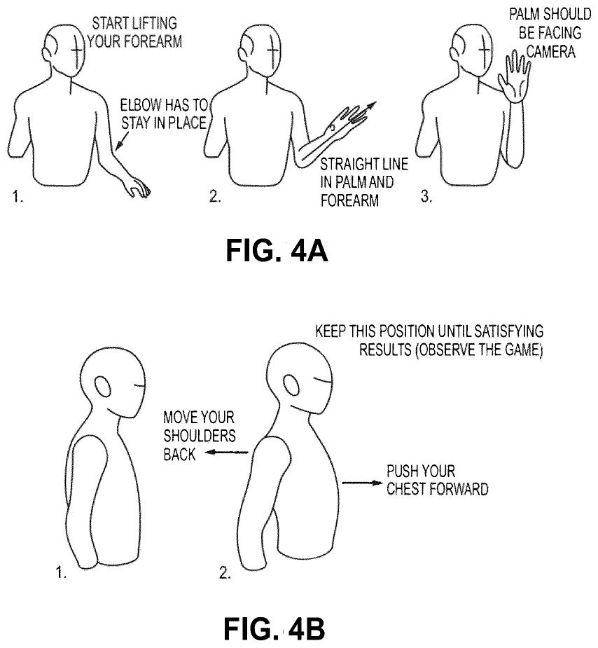

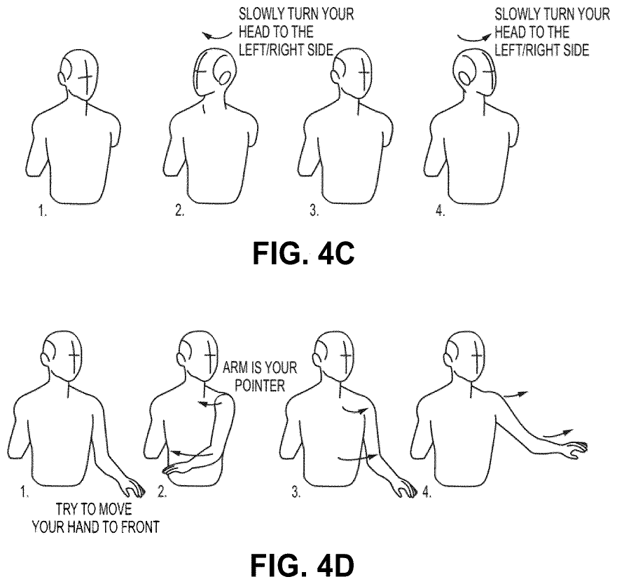

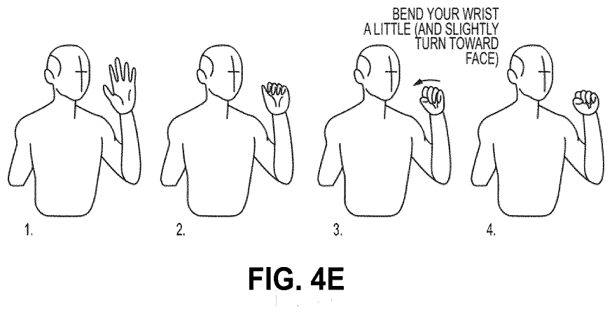

An open and close gesture may be associated with a plurality of predetermined criteria that apply to respective first and subsequent movement data (i.e. criteria that apply to movement data at different time points). A first predetermined criterion may be that the angle between the finger vector (e.g. vector between a hand joint and a hand tip joint) and the palm vector (e.g. vector between the hand joint and a wrist joint) on the same side is within a first predetermined range, or below a first predetermined threshold that is in said first predetermined range. The first predetermined range may be any range identified through a calibration process as described herein. The first predetermined range may be between 30 degrees and 100 degrees. The first predetermined range may be expressed as 65±35 degrees. The first predetermined range may correspond to a closed hand. A second predetermined criterion may be that the angle between the finger vector and the palm vector on the same side (and the same side as that which satisfies the first predetermined criterion) is within a second predetermined range, or above a second predetermined threshold that is in the second predetermined range. The second predetermined range may be any range identified through a calibration process as described herein. The second predetermined range may be between 100 degrees and 180 degrees. The second predetermined range may be expressed as 140±40 degrees. The second predetermined range may correspond to a closed hand. The second predetermined criterion may apply to movement data that is subsequent in time to the movement data that satisfies the first predetermined criterion. In other words, meeting the second predetermined criterion may be conditional on the first predetermined criterion having been met. Thus, an open and close gesture may be identified in movement data where the movement data at a first time point satisfies the first predetermined criterion and movement data at a second time point subsequent to the first time point meets the second predetermined criterion. An open and close gesture may further be subject to one or more additional predetermined criteria that apply at the same time as and/or prior to the first and/or second predetermined criteria. In embodiments, an open and close gesture is further subject to a criterion that applies to the distance between a joint associated with the finger (e.g. hand tip joint) and a joint associated with the wrist (e.g. wrist joint). For example, an open and close gesture can be further subject to a criterion that the distance between a joint associated with the finger and a joint associated with the wrist decreases by more than a predetermined amount. The decrease of the distance between the joint associated with the finger and the joint associated with the wrist can be determined using the ratio of the minimum distance between said joints and a reference value such as the maximum distance between said joints in the movement data (e.g. the movement data that satisfies the first and second predetermined criteria). For example, an open and close gesture may be detected only when said ratio is below a predetermined threshold. The predetermined threshold may be between 0.2 and 0.5, between 0.2 and 0.4, or between 0.3 and 0.4, such as e.g. 0.375. The predetermined threshold may be subject to calibration. The criterion that applies to the distance between a joint associated with the finger and a joint associated with the wrist may be applied at the same time as the second predetermined criterion. Thus, an open and close gesture may be detected in movement data that satisfies that distance criterion, that satisfies the second predetermined criterion and that is subsequent in time to the movement data that satisfies the first predetermined criterion. In embodiments, an open and close gesture is further subject to the criterion that the movement data on the same side as that which satisfies the first and second predetermined criteria satisfies the one or more criteria for an elbow flexion prior to or at the same time as satisfying the first and/or second predetermined criteria. In embodiments, an open and close gesture is further subject to the criterion that the angle between the forearm vector and the upper arm vector on the same side as that which satisfies the first and second predetermined criteria changes from a first value to a second value prior to or at the same time as satisfying the first and/or second predetermined criteria. The first and/or second value may be any value identified through a calibration process as described herein. The first and/or second values may be defined relative to or identical to a first and second predetermined values associated with an elbow flexion. For example, when the first predetermined value associated with an elbow flexion is 100 degrees and the second predetermined value associated with an elbow flexion is 30 degrees, an open & close gesture may be identified when the angle between a forearm vector and an upper arm vector changes from 100 to 30 degrees and the first and predetermined criteria are satisfied at the same time or in subsequent movement data. For example, an open and close gesture may further be subject to the criterion that for the movement data that satisfies both the first and second predetermined criteria, this movement data also satisfies the criteria for an elbow flexion. In embodiments, an open and close gesture may further be subject to the criterion that for one or both of the movement data that satisfies the first criterion and the movement data that satisfies the second predetermined criterion, the angle between a forearm vector and an upper arm vector on the same side as that which satisfies the first and/or second predetermined criteria also satisfies the criterion that this angle is within a range of between 0 and a predetermined value. The predetermined value may be e.g. 40 degrees, 35 degrees, 30 degrees or 25 degrees. The predetermined value may be the same for the movement data that satisfies the first predetermined criteria and for the movement data that satisfies the second predetermined criteria, when the criterion is applied to both. The predetermined value may be the same as or may be defined relative to a second predetermined value (or single value, when a single value is used) used for an elbow flexion gesture. Alternatively, the predetermined value may be different for the movement data that satisfies the first predetermined criteria and for the movement data that satisfies the second predetermined criteria (e.g. 35 degrees and 40 degrees, respectively). Such third predetermined criteria may ensure that the open and close gesture is performed while the subject has their hand up and elbow flexed (as illustrated on FIG. 4E).

The criteria and in particular the choices of vectors and angles described herein may be particularly advantageous in the context of analysing movement of subjects with neuromuscular disorders.

Any method described herein may comprise receiving movement data in real time, i.e. as it is being recorded. In other words, movement data may be analysed by a computing device as described herein as soon as it is received by said computing device. Movement data may be acquired as a series of consecutive frames. The movement data (i.e. each consecutive frame) may be communicated to the computing device by the sensor as soon as it is acquired, and the computing device may analyse this movement data as soon as it is received. In embodiments, the method may not comprise any built-in delay in movement analysis. In other words, accumulating movement data may be analysed as soon as it is received to determine whether a gesture has been performed, without any built-in requirement that a predetermined amount of movement data (e.g. number of frames) has been received prior to the movement data being analysed as described herein. This may in turn ensure that there is no delay in causing an action (e.g. a change in a user interface) to occur when a predetermined movement is detected. Thus, according to any method described herein, movement data may be acquired by a movement sensor (e.g. camera) as the subject, the movement sensor may be operatively connected to a computer system to which the movement sensor sends the data as it is being acquired. The computer system may then analyse the data when it is received to determine whether the movement data satisfies one of more sets of predetermined criteria. In embodiments where a set of predetermined criteria comprise a plurality of criteria that are applied to respective first and subsequent movement data (i.e. criteria that apply to movement data at different time points), the criteria that apply to first movement data may be assessed as soon as first movement data is received, then the further criteria (e.g. second criteria) may be assessed as soon as subsequent movement data is received (optionally only if the criteria that apply to the first movement data were satisfied). The computer system may then trigger a change in the user interface as soon as it is determined that a corresponding set of predetermined criteria has been satisfied. In embodiments where a plurality of sets of criteria may apply, each set (or each first predetermined criteria in a set) may be assessed as soon as movement data is received. In some embodiments the second/subsequent criteria of any set of predetermined criteria comprising multiple criteria for which a first criterion has been satisfied may then be assessed as soon as any further movement data is received. The first criteria of any such set or the first/only criteria of all sets may also be assessed when any further movement data is received.

Treatment of Neuromuscular Disorders with Digital Physiotherapy

The methods described herein treat neuromuscular diseases through the use of a digital physiotherapy tool, optionally in combination with one or more further therapies. In particular embodiments, the present disclosure relates to methods for treating a neuromuscular disease in a subject using a therapeutic compound or composition, in combination with implementing a digital physiotherapy method that comprising: providing a user interface to the subject, receiving movement data comprising coordinates of a plurality of joints of the subject or data derived therefrom at a plurality of time points and in response to receiving movement data satisfying a set of predetermined criteria, triggering a change in the user interface, wherein the change is specific to the set of predetermined criteria satisfied. The present disclosure also relates to methods comprising: providing a user interface to a subject that has been diagnosed as having a neuromuscular disease, receiving movement data comprising coordinates of a plurality of joints of the subject or data derived therefrom at a plurality of time points and in response to receiving movement data satisfying a set of predetermined criteria, triggering a change in the user interface, wherein the change to the user interface is specific to the set of predetermined criteria satisfied. The methods may be for the treatment of a neuromuscular disease in the subject. The therapeutic compound or composition may comprise a disease modifying therapeutic. In particular embodiments, the present disclosure relates to methods for treating a neuromuscular disease in a subject using a disease modifying therapeutic, in combination with implementing a digital physiotherapy method that comprising: providing a user interface to the subject, receiving movement data comprising coordinates of a plurality of joints of the subject or data derived therefrom at a plurality of time points and in response to receiving movement data satisfying a set of predetermined criteria, triggering a change in the user interface, wherein the change is specific to the set of predetermined criteria satisfied. The present disclosure also relates to methods comprising: providing a user interface to a subject that has been diagnosed as having a neuromuscular disease, receiving movement data comprising coordinates of a plurality of joints of the subject or data derived therefrom at a plurality of time points and in response to receiving movement data satisfying a set of predetermined criteria, triggering a change in the user interface, wherein the change to the user interface is specific to the set of predetermined criteria satisfied. The methods may be for the treatment of a neuromuscular disease in the subject.

Generally, the terms “treat”, “treating”, “treatment”, or the like, mean to alleviate (stabilize, reduce, minimise, or eliminate) symptoms, or to reduce, minimise or eliminate the causation of symptoms either on a temporary or permanent basis. In the context of the present disclosure the terms may specifically refer to alleviating one or more symptoms of a neuromuscular disorder.

A disease modifying therapeutic may be any compound or composition that, when administered to a patient with a neuromuscular disease, treats the disease by targeting at least one of the underlying causes of the disease, In the context of SMA, a disease modifying therapeutic may be a compound that increases the amount of SMN protein concentration. In embodiments, a disease modifying therapeutic is selected from nusinersen (marketed as Spinraza, also known as Ionis-SMNrx, PubChem ID 124037382) and risdiplam (marketed as Evrysdi, PubChem ID 118513932).

The digital physiotherapy methods described herein may have a number of beneficial effects, for example compared to the same course of treatment (which may comprise the use of disease modifying drugs or any other intervention, including non-pharmaceutical interventions and the absence of any other medical intervention) without the physiotherapy tool described herein.

Where effects are assessed using case-control studies, these may be assessed using participants randomized into two arms. The first arm may receive the digital physiotherapy intervention plus local standard of care, and the second arm may receive the local standard of care only or local standard of care plus classical physiotherapy (i.e. physiotherapy delivered by a qualified physiotherapist rather than a computer implemented method as described herein). In embodiment, patients in the second arm receive local standard of care plus classical physiotherapy. Patients in the first arm may use a digital physiotherapy tool as described herein to perform a physiotherapy program as described herein. Comparisons may be subject to statistical tests as known in the art, such as for example using a null hypothesis that two arms have the same effect, and alternative hypothesis that the two arms have different effects (two-sided test), with a chosen level of significance (e.g. 0.05).

Subjects receiving the digital physiotherapy intervention may use a digital physiotherapy tool as described herein using a predetermined dosing scheme. A first predetermined dosing scheme may be applied for a first period of time, and a second predetermined dosing scheme may be applied for a second period of time. A first predetermined dosing scheme may be applied for a first subject, and a second predetermined dosing scheme may be applied for a second subject. A predetermined dosing scheme may be determined by a healthcare professional, such as e.g. a physiotherapist. A predetermined dosing scheme may comprise the use of a digital physiotherapy tool as described herein for at least a predetermined amount of time per day with at least a predetermined weekly frequency. For example, a predetermined amount of time per day may be selected from 10, 15, 20 or 25 minutes per day, or more. A predetermined weekly frequency may be selected from 2, 3 or 4 times a week (i.e. on any 2, 3 or 4—or more—days each week), or more such as e.g. up to every day/7 times a week.

Subjects receiving the digital physiotherapy intervention may use a digital physiotherapy tool as described herein for at least 10, 15, 20 or 25 minutes per day, at least 2, 3 or 4 times per week. For example, subjects receiving the digital physiotherapy intervention may use a digital physiotherapy tool as described herein for approximately 15 minutes, approximately 20 minutes or approximately 25 minutes per day or more, approximately 4 times per week or more. Specifically, subjects receiving the digital physiotherapy intervention may use a digital physiotherapy tool as described herein for approximately 15 minutes, approximately 20 minutes or approximately 25 minutes per day, approximately 4 times per week. All times and frequencies may be averages over a predetermined period of time. Subjects receiving the digital physiotherapy intervention may use a digital physiotherapy tool as described herein for at least a predetermined period of time. The predetermined period of time may be 1 month, 3 months, 6 months, 9 months, 12 months, or more. The predetermined period of time may be 4 weeks, 6 weeks, 8 weeks, 10 weeks, 12 weeks, 14 weeks, 16 weeks or more. The predetermined period of time may be 8 weeks.

### Effect on Motor Function

The methods described herein may improve and/or stabilize motor function as assessed using one or more clinical scales and/or one or more patient reported outcome metrics and/or one or more quality of life metrics and/or one or more fatigue metrics, for example compared to treatment without the digital physiotherapy tool. The methods describe herein may improve and/or stabilize motor function as assessed using one or more clinical scales and/or one or more patient reported outcome metrics, compared to treatment with the same standard of care without the digital physiotherapy tool. Treatment with a standard of care regimen may comprise the administration of one or more therapeutics as described herein. Stabilization of motor function may refer to a reduced degradation of motor function over a predetermined period of time, compared to a control patient or population that does not receive the intervention (i.e. treatment without the digital physiotherapy tool). Thus, the methods described herein may improve motor function after a predetermined period of time using a digital physiotherapy tool as described herein, may result in a motor function that is similar or substantially identical after a predetermined period of time using a digital physiotherapy tool as described herein and prior to said use, or may result in a motor function that is reduced after a predetermined period of time using a digital physiotherapy tool as described herein by an amount that is lower than a patient or control population that has not been treated using a method comprising the use of a digital physiotherapy tool as described herein.

According to embodiments of the disclosure, motor function may be assessed using one or more clinical scales selected from: the revised upper limb module (RULM), the Hammersmith Functional Motor Scale (HFMS), and the Motor Function Measure (MFM).

According to embodiments of the disclosure, motor function may be assessed using one or more patient reported outcome metrics or quality of life metrics selected from: the Canadian

Occupational Performance Measure (COPM), the Children's Assessment of Participation and Enjoyment (CAPE), the PedsQL™ 4.0 Generic Core Scales (Pediatric Quality of Life Inventory™), the fatigue severity scale (FSS), the Modified Fatigue Impact Scale (MFIS), and the Visual Analogue Scale for Fatigue (VAS-F), the PROMIS-SF, the SMAIS, and the SMAIS-ULM.

According to embodiments of the disclosure, motor function may be assessed using the revised upper limb module (RULM). The RULM is a test for upper limb function for patients with SMA. It was designed to assess changes in upper limb motor function over time. A description of the RULM can be found in Mazzone et al., 2017 and in the Société Francophone d'Études et de Recherche sur les Handicaps de l'Enfance (SFERHE)/Biogen manual and score card available at mfm-nmd.org/wp-content/uploads/2020/01/TSPIC07_SPINRAZA_Scorecard-RULM.pdf. The scale aims to incorporate functional performance of the shoulder, elbow, wrist and hand. The scale comprises a plurality of tests requiring the subject to perform a predetermined task requiring upper body movement (items A to T), within a maximum of 3 attempts. All tests may be performed while the subject is sat down in front of a table. Each test is scored on a 3-levels scale apart from the first item (item A) which is scored on a 7 levels scale. Each scale starts at 0, with increasing integer numbers corresponding to increased levels of motor function. For example, item B requires the subject to move their hands from their thighs to resting on a table in front of them. A subject obtains a score of 0 if they are not able to move one hand to the table, a score of 1 if they are able to move one hand to rest completely on the table, and a score of 2 if they are able to move both hands to rest completely on the table either simultaneously or one after the other. A single RULM score can be obtained as the sum of the scores obtained by a subject over the plurality of tests. According to embodiments of the disclosure, motor function may be assessed using one or more scores from the RULM, such as scores associated with one or more tests of the RULM or combinations thereof (e.g. the sum of scores associated with a plurality of, including a subset or all of the tests of the RULM).

According to embodiments of the disclosure, motor function may be assessed using the Hammersmith Functional Motor Scale (HFMS). The HFMS is a scale for motor function developed for children with SMA. As used herein, the HFMS refers to the original HFMS as described in Main et al., 2003, or the revised HFMS described in Krosschell et al. 2006. The two scales use the same items but performed in a different order. The scales comprise 20 tests which each require the subject to perform a predetermined task. The order of items for the original HFMS is shown in Table 1 of Krosschell et al. 2006 and the order in the revised scale is shown in Table 2 of Krosschell et al. 2006. Each item is scored on a 3-point scale, with 2 representing the subject being able to perform the task unaided, 1 representing the subject being able to perform the task aided, and 0 representing the subject being unable to perform the task. The scores for each of the 20 tasks are added up, leading to a maximum total of 40.

According to embodiments of the disclosure, motor function may be assessed using the Motor Function Measure (MFM). A description of the MFM can be found in Berard et al., 2005. The MFM is a scale for motor function developed for neuromuscular diseases. The scale comprises 32 items performed in lying, sitting and standing positions. Each item requires the subject to perform a predetermined task. Each item is scored using a 4-point scale. A score of 1 is selected if the subject does not initiate movement or cannot maintain the starting position. A score of 1 is selected if the subject partially completes the exercise. A score of 2 is selected if the subject completes the exercise with compensations, slowness or obvious clumsiness. A score of 3 is selected if the subject completes the exercise with a standard pattern. The total score is obtained by summing the scores over all items, and ranges from 0 to 96. The tasks to be performed in each item are listed in Table 1 of Berard et al., 2005.

According to embodiments of the disclosure, motor function may be assessed using a patient reported outcome metrics, such as the SMA independence scale, or the SMA independence scale-upper limb module (SMAIS-ULM). The SMAIS and SMAIS-ULM are described in Trundell et al. 2022. The SMAIS is a 29-items scale, while the SMAIS-ULM is an upper limb version comprising a subset of 22 items. Each item refers to an activity (e.g. an everyday activity like turning a page, washing hair, picking up an object (, with participants asked to indicate the level of assistance needed to perform the activity, on a scale of 0 to 5:0 (cannot do this at all without help); 1 (need a lot of help); 2 (need a moderate amount of help); 3 (need a little bit of help); 4 (do not need help); and 5 (not applicable [e.g. due to age]). The SMAIS-ULM includes a subset of these that predominantly measure the level of assistance required for activities related to upper limb function.

According to embodiments of the disclosure, motor function may be assessed using one or more variables calculated from one or more wearable magneto-inertial sensors (devices configured to record angular velocities and linear accelerations in 3 directions) placed on the wrists of a subject. For example, metrics such as the mean rotation rate of the wrist, average acceleration, vertical acceleration and power calculated from measurements of the ActiMyo™ device (part of the Syde platform available from SysNav healthcare) as described in Gargaun et al. 2017 have been shown to correlate with clinical metrics such as the Motor Function Measure scale. In embodiments, motor function may be assessed using one or more variables calculated from one or more wearable magneto-inertial sensors placed on the wrists of a subject, selected from 99th percentile upper limb rotational effort and 99th percentile vertical wrist acceleration.

According to embodiments of the disclosure, motor function may be assessed using one or more patient reported outcome metrics, such as the performance metric and/or the satisfaction metric from the Canadian Occupational Performance Measure (COPM). The COPM is an instrument for quantifying a patient satisfaction with an intervention designed to address occupational performance problems. The COPM assesses a subject's perceived occupational performance in the areas of self-care, productivity and leisure. The COPM is described in Law et al., 1990. Two scores, for performance and satisfaction with performance are obtained. The COPM is performed as a five steps semi-structured interview. In a first step, the subject identifies daily occupations of importance to them (e.g. because they want to, need to or are expected to but are unable to perform these activities), within areas of self-care, productivity or leisure. In a second step, the subject rates the importance of each of these occupations to their life on a 10-point scale. In a third step, up to 5 of the most important activities identified in the second step are selected. In a fourth step, the subject rates their own level of performance and satisfaction with performance for each of the 5 activities on a 10-point scale, where 1 indicates poor performance/low satisfaction, and 10 indicates very good performance/high satisfaction. In a fifth step, the rating of step 4 is repeated at a predetermined time after an intervention to be assessed was initiated. These scores are then used to calculate a performance change score and a satisfaction change score. Methods of the disclosure may result in an improvement or stabilization of a performance change score and/or a satisfaction change score according to the COPM in a subject, compared to a subject who is treated without the use of a digital physiotherapy method or tool as described herein. In embodiments, methods of the disclosure may result in an improvement or stabilization of a performance change score according to the COPM in a subject, compared to a subject who is treated without the use of a digital physiotherapy method or tool as described herein. In embodiments, methods of the disclosure result in an improvement of performance and/or satisfaction scores on the COPM by at least two points.

According to embodiments of the disclosure, motor function may be assessed using one or more patient reported outcome metrics, such as the fatigue severity scale (FSS), the Modified Fatigue Impact Scale (MFIS), the PROMIS Short Form (PROMIS-SF), or the Visual Analogue Scale for Fatigue (VAS-F). The FSS is a method for quantifying the impact of fatigue on a subject. The FSS is a self-reported scale of nine items about fatigue, its severity and how it affects daily activities. Each item is scored on a seven-point scale (from 1=strongly agree, to 7=strongly disagree). Thus, the minimum score is 9 and the highest score is 63. Higher scores indicate more severe fatigue and/or fatigue that more strongly impacts the subject's activities. The MFIS is a method for quantifying the impact of fatigue on a subject. The MFIS is a self-reported scale of 21 items quantifying the effects of fatigue in terms of physical, cognitive, and psychosocial functioning. The total score is calculated over all 21 items although subscale scores for physical, cognitive, and psychosocial functioning can also be generated by calculating the sum of specific sets of items. According to the present disclosure, motor function may be assessed using the total MFIS score or the score of the physical subscale of the MFIS. Each item is scored on a 5 point scale, quantifying the patient's agreement with a respective statement, from 0 (never) to 4 (almost always). Thus, the minimum score is 0 and the maximum score is 84. The physical subscale contains 9 items (maximum score 36). The Visual Analogue Scale for Fatigue (VAS-F) is a self-reported scale of 18 items related to the subject's experience of fatigue. Each item asks the respondent to choose a value between 0 and 10 quantifying how they feel between two extreme statements, e.g. I feel not at all tired (0) and I feel extremely tired (10), or place a “X” representing how they currently feel along a visual analogue line that extends between the two extremes. In the latter case, scores for each item are between 0 and 100 and are measured based on the position of the “X” in mm, where the line is 100 mm. The Patient Reported Outcomes Measurement Information System (PROMIS) is a set of standardized tool for measuring PROs. PROMIS-SF refers to either the PROMIS F-SF or the PROMIS parent proxy short form. The PROMIS F-SF is a seven items scale that measures the experience of fatigue and the interference of fatigue on daily activities over the past week (see www.healthmeasures.net/explore-measurement-systems/promis/intro-to-promis). Each item is scored on a 5-point Likert scale, ranging from 1=never to 5=always, in response to questions such as “How often did you feel tired” or “How often were you too tired to take a bath/shower”. Scores range from 7 to 35. The PROMIS parent proxy short form is a 10-item scale investigating perceived fatigue in different situations during the previous week. The grading of each item ranges from 1 to 5, where 1 indicates never, 5 indicates always, and the final score represents the mean value of the 10 items. Methods of the disclosure may result in an improvement (i.e. reduction) or stabilization of a FSS, MFIS, PROMIS-SF or VAS-F score in a subject, compared to a subject who is treated without the use of a digital physiotherapy method or tool as described herein.

According to embodiments of the disclosure, motor function may be assessed using one or more patient reported outcome metrics, such as the Children's Assessment of Participation and Enjoyment (CAPE). The CAPE is a 55-item questionnaire that assesses how children and youth (e.g. subjects between the ages of 6 and 21 years old) participate in daily activities. It is described in King et al., 2004. It provides information about 5 dimensions of participation including diversity of activities done, frequency of participation as a function of the number of possible activities within a category, and enjoyment of activities. The CAPE comprises an overall participation score, which can be separated between a score for formal activities and a score for informal activities, each of which can be further separated between scale scores for five types of activities (recreational, active physical, social, skill-based, self-improvement). Methods of the disclosure may result in an improvement (i.e. increase) or stabilization of a CAPE score in a subject, compared to a subject who is treated without the use of a digital physiotherapy method or tool as described herein. A CAPE score may be any of an overall participation score, a participation score for formal activities, a participation score for informal activities, and a participation score for any of recreational activities, active physical activities, social activities, skill-based activities, and self-improvement activities.

According to embodiments of the disclosure, motor function may be assessed using one or more patient reported outcome metrics, such as the PedsQL™ 4.0 Generic Core Scales (Pediatric Quality of Life Inventory™), The PedsQL™ 4.0 Generic Core Scales is described in www.pedsql.org/PedsQL-Scoring.pdf. The PedsQL™ 4.0 Generic Core Scales may be any of: a child and parent report for young children (Ages 5-7), a child and parent report for Children (Ages 8-12) and a child and parent report for Teens (Ages 13-18). Each of these is composed of 23 items comprising 4 dimensions. The dimensions are: physical functioning (8 items), emotional functioning (5 items), social functioning (5 items) and school functioning (5 items). Higher scores are associated with higher quality of life. Each item is scored on a 5-point Likert scale from 0 (never) to 4 (almost always) or a 3-point Likert scale of 0 (not at all), 2 (sometimes) and 4 (a lot). The 3-point Likert scale is used in the Young child report. The 5-point Likert scale is used in Child and Teens reports. Items are reverse scored and linearly transformed to a 0-100 scale (0=100, 1=75, 2=50, 3=25, 4=0). A mean score is calculated as the sum of the items over the number of items answered, when at least 50% of the items in the scale are answered. Methods of the disclosure may result in an improvement (i.e. increase) or stabilization of a PedsQL™ 4.0 Generic Core Scales score in a subject, compared to a subject who is treated without the use of a digital physiotherapy method or tool as described herein. A PedsQL™ 4.0 Generic Core Scales score may be a score for any one or more (or all) of the 4 dimensions of the scales as described above.

Effects of the methods described herein may be measured by comparing any of the above metrics prior to the use of the methods described (baseline) and after use for a predetermined period of time, such as e.g. 8 weeks or 16 weeks. Effects of the methods described herein may be measured by comparing any of the above metrics or change in said metrics between a group of subjects making use of the methods described herein, and a control group of subjects.

### Adherence and Adoption

The methods described herein may improve adherence to physiotherapy, for example compared to a treatment that comprises a recommendation to perform one or more physiotherapy exercises at a predetermined dosing without the digital physiotherapy tool. The methods described herein may reduce the likelihood of interruption, unsupervised modification (i.e. modification that is not one selected by a qualified physiotherapist), or discontinuation of the physiotherapy. The methods described herein may increase the frequency of performance of one or more physiotherapy exercises. The method described herein may comprise maintaining a predetermined dosing of a digital physiotherapy intervention for a predetermined period of time. Thus, also described herein is a method of treating a subject who has been diagnosed with a neuromuscular disease, the method comprising performing a digital physiotherapy method as described herein, wherein the digital physiotherapy method is performed using at least a predetermined dosing for at least a predetermined period of time. The predetermined period of time may be at least 1 month, at least 3 months, at least 6 months, at least 9 months or at least 12 months.

A predetermined dosing of a digital physiotherapy intervention as described herein may refer to the use of a digital physiotherapy tool as described herein (which is equivalent to the performance of a digital physiotherapy method as described herein) for at least a predetermined amount of time and at least a predetermined frequency. The predetermined amount of time may be at least 10, 15, 20 or 25 minutes per day. The predetermined frequency may be at least 2, 3 or 4 times per week. A predetermined dosing may be defined for a particular subject. A predetermined dose may change during the course of a digital physiotherapy intervention as described herein. For example, a predetermined dose may comprise an amount of time of at least 10 minutes per day at the start of an intervention, and a higher minimum amount later in the intervention. A dose escalation paradigm may be determined as described in Lott et al., 2020 or at the judgment of a qualified physiotherapist based on respective assessments. A predetermined amount of time of use of a digital physiotherapy method as described herein may refer to a total amount of time of use of a digital physiotherapy tool as described herein, or an active amount of time of use of a digital physiotherapy tool as described herein. A total amount of time of use of a digital physiotherapy tool may be defined as the time between the first step of displaying a user interface to the subject according to a method as described herein, and a last step of triggering a change in the user interface according to a method as described herein, wherein the user interface is continuously displayed between said first step and said last step. Said first step may correspond to a start time, such as e.g. when the subject causes the tool to start operating for example by logging into the digital physiotherapy tool. Said last step may correspond to an exit time, such as e.g. when the subject causes the digital physiotherapy tool to stop operating for example by the logging off the digital physiotherapy tool. In embodiments where a total amount of time is recorded as a total amount per period of time, such as e.g. a total amount per day or per week, the total amount of time may be the sum of a plurality of times between a first step of displaying a user interface to the subject according to a method as described herein, and a last step of triggering a change in the user interface according to a method as described herein, wherein the user interface is continuously displayed between said first step and said last step.

An active amount of time of use of a digital physiotherapy tool as described herein may refer to an amount of time associated with movement data that was found to satisfy the one or more predetermined criteria as described herein. For example, when movement data is found to satisfy one or more predetermined criteria as described herein, the period of time between the latest and the earliest of the plurality of time points for which the movement data was found to satisfy the one or more predetermined criteria may be considered to be “active time”. In embodiments where an active amount of time is recorded as an active amount per period of time, such as e.g. an active amount per day or per week, the active amount of time may be the sum of a plurality of times between a first step of periods of time associated with movement data that was found to satisfy the one or more predetermined criteria as described herein. When referring to a physiotherapy that comprises the use of the digital physiotherapy methods and tools as described herein, a total amount of time may refer to the combination of a total or active amount of time of use of a digital physiotherapy tool as described herein and a self-reported or externally measured total amount of time that the subject performs a predetermined set of physiotherapy exercises and/or an undetermined physical activity, for example over a period of time such as a day or a week.

When referring to a physiotherapy that does not comprise the use of the digital physiotherapy methods and tools as described herein, a total amount of time may refer to a self-reported or externally measured total amount of time that the subject performs a predetermined set of physiotherapy exercises and/or an undetermined physical activity, for example over a period of time such as a day or a week. The subject may perform the predetermined set of physiotherapy exercises in guided and/or independent sessions and the total amount of time may refer to the total duration of such a session. The methods described herein may result in a total amount of time of use of the digital physiotherapy methods and tools as described herein that is higher or maintained over a longer period of time than a corresponding total amount of time of performing a physiotherapy that does not comprise the use of the digital physiotherapy methods and tools as described herein. The former may be measured in a first cohort and the latter may be measured in a second cohort. The methods described herein may result in a sustained total amount of time of use of the digital physiotherapy methods and tools as described herein. A sustained total amount of time may be an amount of time that meets at least a predetermined dose over at least a predetermined period of time. This may be administered in addition to a self-reported amount of time of physiotherapy that does not comprise the use of the digital physiotherapy methods and tools as described herein. Thus, a first cohort may be provided with access to the digital physiotherapy methods and tools as described herein and an optional second cohort may not be provided with access to these tools. The first and optional second cohorts may be provided with access to a self-reported amount of time of classical physiotherapy. Thus, any amount of time of use of the digital physiotherapy methods and tools as described herein may be considered to be an increase in the total amount of physiotherapy time.

### Subject

As used herein, the term “subject” is a human subject. The subject can be a subject who has or is at risk of developing a neuromuscular disorder. For example, the subject may have been diagnosed as having spinal muscular atrophy (SMA). The subject may have been diagnosed as having SMA type I, SMA type II or SMA type III. The subject may have been diagnosed as having SMA type II or SMA type III. In embodiments, the subject has been diagnosed as having SMA type II.

The subject may have a neuromuscular disorder selected from: a motor neuron disease (such as e.g. amyotrophic lateral sclerosis), a toxic neuropathy, a congenital myopathy, a muscular dystrophy (such as e.g. Duchenne muscular dystrophy, Becker muscular dystrophy, congenital muscular dystrophy, and facioscapulohumeral muscular dystrophy), a metabolic myopathy, a neuromuscular junction disorder (such as e.g. myasthenia gravis or Lambert-Eaton Syndrome), and spinal muscular atrophy (such as e.g. type I, type II or type III SMA). The subject may be a subject who has been diagnosed as having type II or type III SMA.

The subject may be a subject who has been or is being treated with one or more therapeutics. A therapeutic may be any compound or composition for treating a neuromuscular disease or disorder. For example, a therapeutic may be selected from nusinersen, risdiplam, and onasemnogene abeparvovec (also known as Zolgensma, which is a gene therapy medication that increases SMN protein concentration by delivering a SMN1 transgene).

The subject may be a subject of at least 6 years old. The subject may be at least 8 years old. The subject may be a pediatric subject. The subject may be aged between 6 and 18 years old or between 8 and 18 years old. The subject may be an adult. In embodiments, the subject is a subject between 6 and 21 years old, between 6 and 18 years old, or between 8 and 18 years old. The subject may be an ambulant subject or a non-ambulant subject. The subject may be a subject who is able to stay seated independently for at least 10 seconds. The subject may be a subject who has a score of at least 2 points in entry item A of the RULM (i.e. “Can raise 1 or 2 hands to the mouth but cannot raise a 200 g weight in it to the mouth”). The subject may be a subject who is undergoing treatment with a disease modifying therapy. The subject may be a subject who has been undergoing treatment with a disease modifying therapy for at least 6 months. The subject may be a subject who has a confirmed genetic diagnosis of 5q SMA.

All publications mentioned herein are incorporated by reference in their entirety.

### EXAMPLES

The invention is further illustrated by the following examples. It will be appreciated that the examples are for illustrative purposes only and are not intended to limit the invention as described above. Modification of detail may be made without departing from the scope of the invention.

### Example 1: Clinical Trial Evaluating the Effect of a Digital Physiotherapy Tool on Patients with Spinal Muscular Atrophy

### Introduction

The nature of how best to promote physical activity in patients with SMA is evolving, with the introduction of interactive computer-based platforms such as active video-based exergames. These are commonly defined as applied computer applications designed primarily for a purpose other than mere entertainment, including training, education and health care. The use of active videogames for rehabilitation (exergaming) has a positive impact on motivation towards training, is flexible in scheduling at home to allow higher dosing, and has proven useful to enhance strength, coordination and mobility. Evaluating the potential synergistic benefit of low-cost interventions such as those designed to promote increased physical activity has strong potential to inform care and improve outcomes for children with SMA who are receiving disease-modifying drugs and is important to maximize the impact of public investment in expensive orphan therapies.

### Study Protocol and Objectives

A study was conducted to develop and pilot test the intervention that will be incorporated in an investigator initiated trial starting the following year. This future trial will compare the effectiveness of a home-based exergaming intervention as an adjunct to disease-modifying therapy, versus disease-modifying therapy with usual care in improving motor outcomes and self-reported recreational participation among children with SMA.

The project uses the Microsoft Azure Kinect Developer Kit (DK) as the exergaming platform, with advanced artificial intelligence depth sensors and spatial microphone array with a video camera and orientation sensor, capturing the full body movements of a person in a 3D space. Accessible at home, it allows the user to interact directly with a computer-simulated environment allowing players to connect remotely for interactive play. The Kinect platform has been used in medical applications such as physical therapy for individuals with neurological disorders and Parkinson's disease, and gait analysis for multiple sclerosis. Its feasibility as a digital biomarker has been shown in individuals with SMA by Roche's data scientists (see Chen et al. 2017). Home-based exergaming offers the advantage of frequent use with less disruption to school and work schedules, and increases motivation.

During the 1st study phase, a Kinect-based game was newly designed targeting home-based physical activity movement. This was informed by patient, caregiver and physiotherapist preferences, adaptive gaming experts and data analysis (see Example 2 below). The game was initially pilot tested in >12 subjects (patients+age matched controls) with initial instructions at a central office (2 sites) setting followed by 4 weeks at home game play to improve its user interface (see Example 3 below) before initiating the randomized trial in phase 2 with a larger study population.

A multisite randomized registry-based waitlist controlled trial will be conducted to determine if the exergame Tales from the Magic Keep (see Example 2) is more effective than usual care for improving occupational performance and satisfaction. Secondary outcomes will determine the effects of this exergame compared to usual care on fatigue, daily activities, motor activity, and motor function immediately post-intervention at 8 weeks and post-baseline at 16 weeks following open-label extension for an additional 8 weeks.

In particular, an exergaming intervention will be implemented in a home setting to improve motor functions and patient-reported outcomes for patients with Spinal Muscular Atrophy (SMA), compared to standard of care, and both on top of the Disease Modifying Therapy (DMT) in a randomized clinical trial. A broad patient spectrum with respect to age, gender, physical capability and location will be included. In total ~22 patients with type 2 or type 3 SMA between 8 and 18 years old across 5 sites in Canada will be recruited.

Impaired motor function remains an important cause of functional limitation among children with SMA. Measuring the impact of interventions on motor outcomes is challenging, as existing standardized assessments are neither sensitive to change over short periods of time nor adapted for the full spectrum of abilities in SMA; therefore, they may not reflect clinically important change. Furthermore, standardized motor assessments performed in the clinical setting are subject to patient motivation and performance variation on a given day, are time consuming, and require highly specialized personnel to administer.

In the intervention arm of the trial, the patients should play the exergaming at least 20 mins per day, 4 times per week for 8 weeks, optionally with an additional 8 weeks extension. The patients will be assessed by established clinical rating scales as well as digital tool Syde to measure the wrist movement. In particular, the effect of the intervention on results of clinical rating scales such as the Revised Upper Limb Module (RULM) will be assessed, as well as patients' adherence to the dosing of the intervention scheme.

The data recorded by the exergaming platform will be uploaded to an online secure file system and saved there as the unique copy (no data saved on the local disk after uploading). One game session defined by using EXIT to end the game successfully has one recorded file. The Kinect sensor records whole body movement data in a 3D coordinate system as the locations and rotations of 32 joints, in real time. The following data will be recorded. The recorded file has three parts of data: metadata, sensor data, and game data. The metadata comprises: an anonymised patient identifier (PatientID), a login time (in a common time coordinate system, such as UTC), a logout time (in the common time coordinate system), and one or more calibration settings providing angular conditions for each gesture. The calibration settings are obtained by prompting the subject to perform each gesture (e.g. by showing a character on a user interface performing the gesture) before starting the game the 1st time, to calibrate the expected angles for each gesture, so the system can recognise the gesture as performed by the subject. A tolerance may be added around the angles detected at calibration, such as e.g. 5-10%. The subject may be prompted to perform the gesture once or multiple times (e.g. depending on the type of gesture) for which an average or minimum/maximum angles may be selected. The sensor data comprises data recorded directly by the Kinect device, including: NumberofSkeletons, SkeletonID, TimeDevice, and data for each of the 32 joints detected including a joint identifier (JointID), Position (x, y, z), Orientation (x, y, x, w—where x, y, z is an orientation vector and w is a scalar that stores the rotation around the vector), and ConfidenceLevel. The NumberOfSkeletons is the number of bodies detected by the Kinect sensor. Kinect is able to detect up to 6 bodies in parallel. In the present method, if more than 1 body is detected, a warning message may be issued. The system may still continue to work with the first body detected. The SkeletonID is the identifier associated with each detected body. This value is assigned automatically by Kinect and is also based on the number of detected bodies. The TimeDevice is the time value of the Kinect device. This value might be different from the System (computer) time value as the Kinect sensor has its own time system. The Kinect sensor assigns one of 3 confidence levels to joint data: none, low and medium. In the present examples, >90% of the detected joints were found to have the highest confidence level (medium). The confidence level may be ignored for gesture detection (as in most cases the confidence level is on the highest level). The confidence level may be used for debug purposes (e.g. when an issue is detected in the game). The game data comprises data characterizing the player's progression in the game, and includes: ScreenLabel, PotionsNum and Performance. ScreenLabel is the current state of the application (e.g. main menu, Lark's room, Alchemy room). PotionsNum is the number of potions performed in the current session. Performance is the number of times a gesture is performed in the current session.

Physio data may also be recorded, comprising data calculated from the sensor data. The physio data comprises data derived from the sensor data and characterizing the game gestures performed during the game play, and includes: Angular conditions for each gesture per frame, a GestureLabel, and other derived metrics. At least part of the physio data may be calculated offline (i.e. after a game session is terminated).

The data file may also contain gesture conditions. Gesture conditions may comprise a plurality of values that are relevant to evaluation of predetermined criteria for each of the gestures used. For example, a gesture condition can be be: OC_HandLeftToHandTipLeftRotation (many gesture condition may be available). A value for each gesture condition (e.g. angle in degrees) may be associated with every frame. Gesture conditions may further comprise data fields indicating, in each frame, where a particular gesture predetermined criterion or set of criteria is fulfilled. For example, one gesture could be HA-L (Horizontal Abduction Left). Every frame may be associated with a value for this gesture: for example 0 may mean “gesture not fulfilled”, 1 may mean “gesture fulfilled”.

Adherence to the exergaming intervention will be characterized by quantifying the game duration (time between logout and login) and movement duration (time with active movement during the game duration) in each session. Time with active movement can be derived from the gesture conditions information and the time stamps associated with the frames. Adherence to the exergaming intervention may be compared to adherence to a comparative physiotherapy programme that does not rely on the exergame. For example, the total movement duration per week may be compared between a first group that undergoes physiotherapy using the exergame and a second group that undergoes a combination of self-supervised physiotherapy at home and guided physiotherapy. The total movement duration per week may only capture movement associated with the exergame. Thus, adherence may be measured in terms of additional physiotherapy time that is associated with the exergame and that would not be performed in the absence of the exergame.

The effect of the exergaming intervention on the subject's motor function will be assessed using one or more clinical scales or combinations thereof. For example, motor function may be assessed using the revised upper limb module (RULM).

Further, the effect of the exergaming intervention on the subject's motor function will be assessed using digital endpoints. Digital endpoints captured by a sensor worn unobtrusively in their home environments during activities of daily living may provide more precise and objective indicators of motor change relative to measures in clinical settings such as the RULM. This data from long wear periods can be synthesized into meaningful aggregates, providing a more complete picture of abilities. Syde® is the first digital endpoint qualified by the European Medicines Agency in ambulatory Duchenne muscular dystrophy, meeting regulatory criteria for analytic and technical validity and for content and construct validity. It has since been used in several studies in rare disease populations, including SMA (where it has shown excellent test-retest reliability), with a high potential for validation of upper limb endpoints in non-ambulant patients.

The primary hypothesis that will be tested is that for youth with SMA aged 8-18 years, the methods described herein (Tales from the Magic Keep) will be more effective than usual care immediately post-intervention at 8 weeks in improving performance and satisfaction scores on the Canadian Occupational Performance Measure (COPM) by at least a clinically meaningful difference of two points.

The secondary hypothesis tested will include one or more of the following. A first secondary hypothesis is that for youth with SMA aged 8-18 years, the methods described herein will be more effective than a waitlist control group receiving usual care immediately post-intervention at 8 weeks in terms of one or more of: (i) reducing fatigue as measured on the Patient-Reported Outcomes Measurement Information System (PROMIS-SF), improving independence in activities of daily living as measured on the SMA Independence Scale (SMAIS), and improving the peak motor activity as measured by upper limb outputs from the Syde. The Syde® digital endpoints of change in 99^{th} percentile upper limb rotational effort and 99th percentile vertical wrist acceleration in particular will be assessed.

Participants with SMA on DMT will be randomized to one of two arms: (1) exergaming intervention and usual care or (2) waitlist usual care alone. Usual care will be defined as following the established consensus care guidelines. The planned dosing of the intervention is a minimum of 20 minutes, 3 times per week over 8 weeks (8 total hours). An 8-week period was chosen to reflect typical intervention blocks offered in rehabilitation to strengthen motor performance. A 20-minute session was chosen to minimize the potential for fatigability in youth with SMA. The primary outcome is measured immediately post-intervention at 8 weeks; participants in the waitlist control group will then have an opportunity to access the exergame during the open-label phase, giving all participants an opportunity to play.

Inclusion criteria will include that the participants must meet the following eligibility criteria prior to randomization: 1) Confirmed genetic diagnosis of 5q SMA; 2) Aged 8-18 years old; 3) Able to stay seated independently without support for at least 10 seconds; 4) A score of at least 2 points in entry item A of the RULM (i.e., “Can raise 1 or 2 hands to the mouth but cannot raise a 200 g weight in it to the mouth”); 5) Treated with disease-modifying therapy (on an approved disease-modifying therapy for at least 6 months.) and 5) Provide consent. Exclusion criteria include: 1) Inability to comply with study procedures according to the site investigator, 2) Severe scoliosis or contractures that would interfere with gameplay or with successful completion of functional assessments, as confirmed by the clinical evaluator, and 3) planned orthopedic surgery 6 months prior to or throughout intervention and follow-up period.

For the intervention group, one in-person calibration and an optional recalibration will be performed at the study site. Participants will receive weekly virtual check-ins with the study physiotherapist/research coordinator for troubleshooting and motivational purposes. Adherence will be both self-reported using a gameplay tracking form and captured digitally directly from the Kinect sensor. Monthly meetings will be organized between trainers of all sites for troubleshooting.

There will be three time points for measurement of all outcomes: baseline (T1), immediately post-intervention primary endpoint at 8 weeks (T2), and at 16 weeks post-intervention (T3). There will be an additional measurement of select outcomes available through the registry at 12 months post-randomization (T4).

The primary outcome is a participant (or proxy)-reported perception and satisfaction with current occupational performance at 8 weeks post-randomization, as measured on the COPM. COPM administration yields two scores: (a) perception of current performance (COPM-P) and (b) satisfaction with current performance (COPM-S), each ranging from 1 (poor performance or lowest satisfaction) to 10 (excellent performance or high satisfaction). A 2 or more-point change in scores between administrations is considered clinically meaningful. The COPM will be administered by a trained occupational therapist at 3 time points: Baseline (T1), 8 weeks (T2, primary outcome) and 16 weeks (T3). Relying on the COPM as the primary outcome and aiming to detect a minimum difference of 2 points, which is considered a meaningful difference between groups at 8 weeks post-randomization, a standard deviation of 1.8, a correlation between baseline and 8 weeks of 0.6, two-sided alpha=0.05 with 80% power, we would require 18 participants (9 per group). To plan for a potential 20% dropout, we will recruit 22 participants (11 per group).

The secondary outcomes include upper limb motor activity metrics defined derived from wearable devices, and in particular using Syde® (Sysnav, France), a class 2 medical wearable device specifically designed for neuromuscular disorders. The Syde® platform includes two wearable sensors for precise movement capture. The sensor is composed of a tri-axial accelerometer, a gyroscope and a magnetometer, providing an objective measure of physical activity obtained from the patient's performance of daily activities in their usual environment. The system is sufficiently light (38 grams), waterproof, and non-intrusive (wireless) to be worn daily over four weeks on the dominant wrist as a watch with a second piece either on the wheelchair or ankle. A three-week daily wear period is sufficient to account for variability in computed variables and to capture aggregate peak performance during this time period. To ensure the reliability of data, record Syde® data will be recorded at three timepoints (Baseline T1, Week 4-8 for T2, and Week 12-16 for T3) with 4 weeks of data collection at each timepoint. Syde® derived digital endpoints representing upper limb activity, including upper limb rotational effort and vertical wrist acceleration will be used. The median and 99th percentile of each variable will be analyzed to determine a patient's typical pattern and their maximum ability during the four-week wear period.

The secondary outcomes include independence in activities of daily living, measured using the SMAIS. The SMAIS is a tool designed to measure the level of daily assistance required by individuals with Type 2 and non-ambulatory Type 3 SMA to carry out their typical daily activities. It is a patient-reported outcome measure for individuals aged 12 years or older who have SMA, and an observer-reported outcome measure for caregivers of those aged 2 years or older. The SMAIS-ULM comprises 22 items that mainly measure the level of assistance required for activities related to upper limb function. The SMAIS will be administered at screening, 8 weeks, and 16 weeks.

The secondary outcomes include fatigue, measured using the PROMIS parent proxy short form, which includes 10 items investigating perceived fatigue in different situations during the previous week. The grading of each item ranges from 1 to 5, where 1 indicates never, 5 indicates always, and the final score represents the mean value of the 10 items. The PROMIS will be administered at screening, 8 weeks, and 16 weeks.

At the baseline visit, participants will receive an in-lab demonstration and assisted calibration of the exergame, and will conduct baseline assessments. During the active phase (weeks 1-8), those in the intervention arm will be asked to play the exergame a minimum of three times a week for 20 minutes over 8 weeks (8 hours total). at week 4 in-lab visit, all participants will be invited for an in-lab visit to wear Syde® for a second measurement period, worn over 4 weeks. At the week 8 in-lab visit (end of primary study T2), all participants will return for an in-lab visit to return the Syde and repeat all primary and secondary outcome assessments. The participants in both arms will then be given the option to enter the open-label period for 8 weeks. At the week 12 in lab visit, all participants will be provided with Syde® to wear for 4 weeks. at the week 16 in-lab visit, the primary and secondary blinded outcome assessments will be repeated at 16 weeks post-randomization. 12 months of follow-up data will be captured from usual care through the SMA Registry.

Patient demographics and baseline outcome variables (both primary and secondary) will be summarized using descriptive summary measures. The primary analysis for both primary and secondary outcomes will rely on the Per-Protocol populations (randomized populations excluding those randomized to Kinect who did not receive the intervention), as our primary goal is to assess the efficacy of the intervention in ideal circumstances. Secondary analyses will include: (i) the intention to treat (ITT) randomized populations and (ii) the as-treated population, which will incorporate trial participants per treatment they received (excluding participants who used the intervention less than the minimal 20 minutes three times a week over 8 weeks as measured on the Kinect). The primary ITT analysis for primary and secondary outcome measures will use Analysis of Covariance (ANCOVA) to test differences in the continuous outcome scores for COPM (and for secondary outcomes) between exergaming vs usual care at 8 weeks (post-intervention), adjusted for baseline values. Hypothesis tests will be two-sided, with a statistical significance of alpha=0.05.

Adherence to the exergaming intervention based on the calculated Game Duration and Movement Duration will be quantified from the Kinect data. Additional planned analyses will include repeated measurements at T1, T2 and T3 and use least square mean differences from the model (with 95% CI) to evaluate the effect of waiting and whether intervention effects in the intervention arm are sustained T3. These analyses will account for correlation in repeated measures on the same participant using a suitable correlation structure.

### Example 2: Co-Creation of a Playful Digital Intervention for Neuromuscular Diseases

The objective of this work was to co-create with knowledge use partners a novel and patient-oriented active videogame (exergame) facilitating home-based physiotherapy for children/youth with neuromuscular diseases. In addition, the work aimed to identify the key considerations to design and develop a safe therapeutic exergame.

Interactive computer-based platforms such as active exergames can be designed and used for healthcare including training and education, other than for mere entertainment. However, the design of exergames for patients with physical impairments that matter to them requires specific considerations such as accessibility, movement limitation, and risk mitigation.

This work adopted a co-creation process that is reflected in both design and development phases. During design, online surveys and semi-structured interviews were conducted with patient groups, physiotherapists and an INFORM Rare SMA Working Group (www.informrare.ca) to identify the needs, preferences, and barriers to implementation. During development, iterative cycles including usability testing by physiotherapists were performed, in order to ensure physiotherapy exercises can be transformed into safe and effective virtual exercises. Game elements were introduced to ensure exercises are fun, intuitive to perform and improve patient engagement in the therapy. In particular, the game was designed as an imaginary world in which the subject has to use their hands to select, add and mix ingredients to make potions.

Crucial features such as accessibility barriers and engagement factors were identified as key considerations for technology adoptability and game design.

The game was designed to use a Kinect device connected to a screen. The user is expected to be sat at a distance of approximately 150 cm from the Kinect device (distance from chest to camera). The game is programmed to show an icon on the screen (e.g. an open eye) when the user is at the right distance from the camera. The icon may disappear or change (e.g. change colour, show a closed eye or a squinted eye) when the user is too close or too far away from the camera, or the light conditions are suboptimal for movement detection. An error message may further be displayed to indicate the likely cause of poor detection, such as e.g. a message indicating that the subject is too close to the camera, that the subject is too far to the left/right of the camera, that the camera is too high or too low, etc. The game is designed for the Kinect device to be located approximately at the height of the subject's navel (approximately 5-10 cm above the waist) in use, when the user is sat down. For a standard chair with a seat approximately 45 cm from the ground, the Kinect device is expected to be at a height of approximately 70 cm from the ground.

Interactions with the game are performed using the body tracking technology implemented in the Kinect device. The Kinect sensor records whole body movement data in a 3D coordinate system as the locations and rotations of 32 joints, in real time. The joints used in the game are illustrated on FIG. 3B. These only include upper body joints, as the game is designed to be usable for users of various abilities including users with limited lower body mobility. The joints used include the following: elbow 30, wrist 31, shoulder 32, head 33, neck 34, spine_chest 35, spine_navel 36, pelvis 37, clavicle 38, hand 39, hand_tip 40. These are used to define a series of vectors that are used to detect gestures (as will be explained further below). Only the left side vectors are shown on FIG. 3B, but right side equivalents exist for all but the head, neck and spine joints.

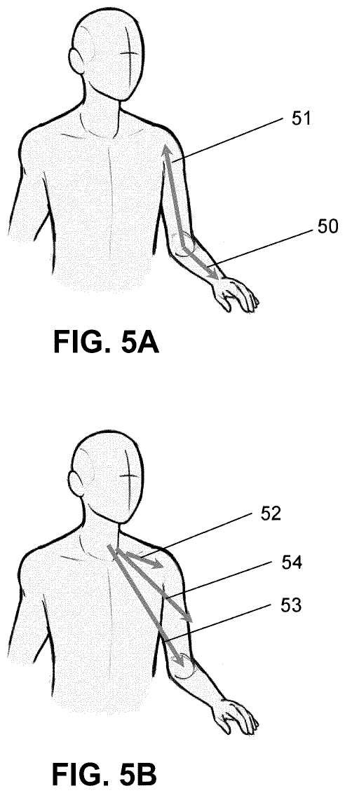

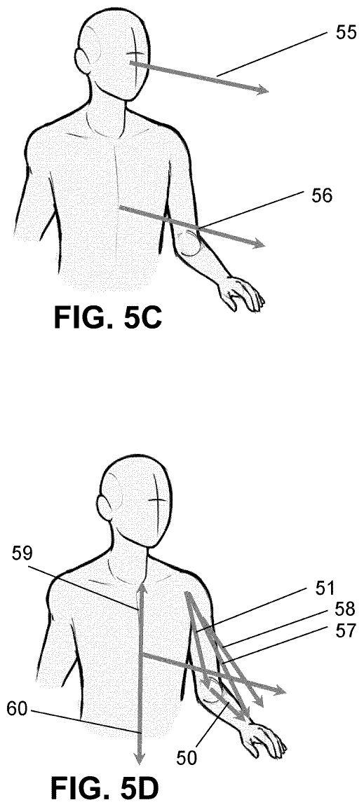

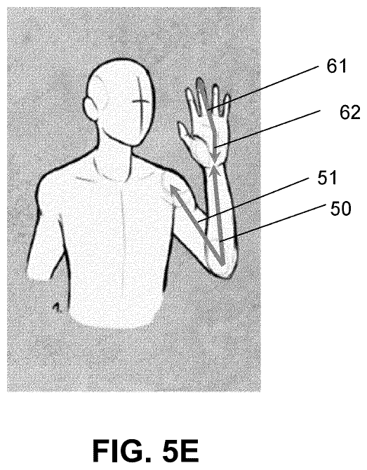

Five primary exercises were selected based on the physiotherapy goals within the targeted patient population. The exercises are illustrated on FIGS. 4A-4E, and include performing an elbow flexion gesture (FIG. 4A), a thoracic extension gesture (FIG. 4B), a head rotation gesture (FIG. 4C), a horizontal abduction gesture (FIG. 4D), and an open-close hand gesture (FIG. 4D). The detection of each of these gestures is illustrated on FIGS. 5A-5E. The elbow flexion and horizontal abduction are single gestures, and the head rotation, thoracic extension and open close are sequential gestures. Single gestures are also referred to herein as “continuous gesture” are detected by detection of a single continuous motion. By contrast, “sequential gestures” involve the detection of multiple gestures in succession. The system is designed such that the second condition of a sequential gesture will necessarily become true eventually (e.g. with a head rotation to the left the subject will always eventually turn the head back to face the sensor). This enables the method to look for fulfillment of the second condition as soon as the first condition is fulfilled without imposing any speed of movement requirement (timeframe for search of the second condition). As illustrated on FIG. 5A, the elbow flexion (EF) gesture is identified as a single gesture involving an angle of between 30 (subject to calibration) and 100 degrees (in 3D, subject to calibration) between the forearm vector 50 (vector between the elbow and wrist joints) and the upper arm vector 51 (vector between the elbow and shoulder joints). As illustrated on FIG. 5B, the thoracic extension (TE) gesture is a sequential gesture that is identified as a succession of a first sequence where the angle between the left and right chest vectors (where a chest vector is a vector 54 obtained as the average between a vector 52 between the clavicle and shoulder joints and a vector 53 between the neck and elbow joints-FIG. 5B only shows the vectors used to calculate the left chest vector) is within 190±12 degrees (subject to calibration), and a second sequence where the same angle is within 160±15 degrees (subject to calibration). Calibration may refer to calibration of the expected value. The tolerance around the expected value may be fixed. As illustrated on FIG. 5C, the head rotation (HR) gesture is identified as a succession of a first sequence where the angle between the head forward vector 55 (the vector from the head joint pointing forward) and the torso forward vector 56 (the vector obtained as the average of the vectors pointing forward from the following joints: neck, spine_chest, spine_navel, pelvis) is within 42±5 degrees, and a second sequence where the same angle is within 0±10 degrees. As illustrated on FIG. 5D, the horizontal abduction (HA) gesture is a continuous gesture identified as the angle between the torso forward vector 56 and the arm vector 58 (which is a vector obtained as the average between the following vectors: shoulder to elbow vector 51 (upper arm vector), elbow to wrist vector 50 (forearm vector), wrist to shoulder vector 57) projection on the transverse plane (see FIG. 3A) is between 0 and 70 degrees (subject to calibration), provided that the following two conditions are met: (i) the angle between the torso down vector 60 (which is a vector obtained as the average of the vectors pointing downward from the following joints: neck, spine_chest, spine_navel, pelvis) and the upper arm vector 51 is between 30 and 80 degrees, and (ii) the angle between the torso up vector 59 (vector obtained as the average of the vectors pointing upward from the following joints: neck, spine_chest, spine_navel, pelvis) and the forearm vector 50 projection onto the transverse plane is within 95±18 degrees. As illustrated on FIG. 5E, the open close (OC) gesture is a sequential gesture identified as the succession of a first sequence in which the angle between the finger vector 61 (vector between the hand and hand_tip joints) and the palm vector 62 (vector between the hand and wrist joints) is between 30 and 100 degrees (subject to calibration) and a second sequence in which the same angle is between 100 and 180 degrees (subject to calibration), provided that the following condition is met: the angle between the forearm vector 50 and the upper arm vector 51 satisfies the criteria for an elbow flexion (i.e. the elbow has been flexed prior to detecting both sequences above).

All gestures are detected in real time. Thus, every frame is labeled with a movement such as e.g. Sequence 1 of thoracic extension, elbow flexion, etc. and an action in the game is triggered by the detection of any frame that is labeled as a single gesture, or any frame that is labeled as the last sequence of a sequential gesture for which the preceding sequence(s) have been identified.

Note that the vectors described above could be identified using different joints/vectors, if using a movement detection system that detects other joints (e.g. using a system other than Kinect).

The game “Tales from the Magic Keep™” was co-created by transforming the exercises requirements into a sequence of movements to be executed in order to acquire alchemy skills in an immersive virtual ancient land through which the player could create new items and explore, as expressed as the main preferences by the patient community.

The elbow flexion is performed in the exploration stages of the game, for example in the main menu to highlight a selected item, or in gameplay to select an ingredient to add to the cauldron. For example, by flexing the elbow, the user can select a single ingredient that will then show as moving to a position above the cauldron. From this position, the user can perform an elbow extension by bringing the forearm down to drop the ingredient in the cauldron. This will cause the game to show the ingredient as dropped into the cauldron. In some cases, the user can repeat the elbow extension to add multiple drops of the same ingredient. In some cases, the ingredient can be pourable (e.g. liquid or uncountable ingredient) and the user can control the pouring using the elbow flexion: extension of the elbow will cause the ingredient to keep pouring, and bringing the forearm up will cause the pouring to show as stopping or slowing down. The EF gesture is ideally performed by the user bending their arm with the palm facing out, and pressing the wrist close to the shoulder without blocking the shoulder (see FIG. 4A).

The thoracic extension gesture can be used during gameplay, to cause the mixture in the cauldron to be stirred, to increase or decrease the density of the potion in the cauldron. The TE gesture is performed by the user pushing their torso to the front as much as possible, and at the same time pushing their shoulder blades together and bringing their arms back (see FIG. 4B).

The head rotation gesture can be used by a user to increase the temperature of the potion. The HR gesture can be performed by the user turning their head to the right (or left) and returning to the front (see FIG. 4C).

The horizontal abduction gesture can be used as a basic movement performed for moving through the game, such as for example to change the position of the cursor in a menu or select objects in 3D. For example, HA can be used in the game menu to choose an item in a list presented (e.g. Play—Settings—Change player—Calibration—Exit), in the “home” play scene to choose the next step of the game by selecting parts of the room shown, in the “Alchemy book” (main menu for levels/challenges to be performed) to select potions or chapters, and in the main gameplay scene (laboratory) and calibration menu to select ingredients. The HA gesture is performed by the user moving the whole arm with the shoulder in a certain direction. The user's elbow is bent while the forearm is horizontal, with the arm at the level of the chest so that the user can move the arm in the horizontal plane to their left or right. The movement can be done by both arms. The elbow is ideally not strictly held against the user's body and instead is at a slight distance, with the forearm perpendicular to the body (see FIG. 4D).

The open and close gesture can be used to proceed with an already selected item or user interface object (e.g. a button), to trigger a specific action. It can be used in a similar manner to double clicking or pushing enter on a conventional computer user interface. The gesture is performed by the user holding their palm facing out while their elbow is flexed. The user's hand goes from fully open to making a first and opening the hand again (see FIG. 4E).

Table 1 below summarises the gestures and where they are used in the current implementation.

The sequence of choice of gestures was determined by the top ranking physiotherapy goals determined by patient/caregiver and physiotherapist surveys. For those, physiotherapists on the project determined 5 gestures that fit these goals and could be performed by the envisioned patient population.

Then game developers designed the game actions to fit those gestures into game movements. The game is divided into 9 chapters wherein chapter 1-3 introduce the 5 gestures with repetitions to train the subject. Then these are followed by 2 groups of specialized chapters with different compositions thereof that have each different focus and could be used for individualized focus (chapter 4 involves more mixing=thoracic extension, chapter 5 involves more heat change=head rotation, chapter 6 involves more drop ingredients=elbow flexion). Thus, the methods described herein may advantageously enable tailoring of physiotherapy exercise to a particular subject by adapting game play to require more or less of any one or more of the predetermined gestures.

TABLE 1
Summary of gestures and uses.

| | Horizontal | Elbow | Open & | Head | Thoracic |
|---|---|---|---|---|---|
| | abduction | flexion | close | rotation | extension |
| Main menu | Yes | Yes | Yes | No | No |
| Lark's room | Yes | Yes | Yes | No | No |
| (home | | | | | |
| scene) | | | | | |
| Laboratory | Yes | Yes | Yes | Yes | Yes |
| (Gameplay) | | | | | |

The main menu enables the user to select from a plurality of displayed options (in particular: calibration, settings, start, change player, exit) by moving a cursor onto an option then selecting it. The user moves the cursor using HA, which causes objects to be highlighted. The EF gesture can then be used to select the object (also referred to as “focus” the object), then the open and close gesture can be performed to confirm the selection. This sequence can be performed every time an object is to be selected.

The calibration process ensures that the gestures are adapted to a user's particular physical abilities. Calibration may be run automatically the first time that the game is launched. A character may be displayed on screen to show the user the gesture to be performed. The user may therefore be prompted to adopt a particular posture (e.g. that adopted by the character displayed on screen), and the positions of the relevant joints, derived vectors and/or angles between derived vectors may be recorded while the subject is holding the posture. A user can select the calibration option in the main menu to recalibrate at any point. A recalibration may be automatically triggered based on gesture data or data derived therefrom (e.g. change in gesture data). In embodiments, the calibration of a gesture may require the subject to fulfill the same posture multiple times (such as e.g. for the elbow flexion gesture) and an average value across these may be used. In embodiments, the calibration of a gesture may only require the subject to fulfill the same posture once (such as e.g. for the HA, HR, TE and OC gestures).

Selecting “start” from the main menu may take the user to the “home” scene, also referred to as “Lark's room”. From the home scene, the user can configure their character, select challenges to be performed, etc. The home scene is explored in a similar manner as the main menu, using HA to navigate, EF to select an item, and OC to confirm a selection. One of the selectable items in the home scene is the Book of Alchemy. This is itself a menu that displays challenges that the user has already completed (potions made) and challenges that the user can select to perform. Game statistics such as scores associated with the challenges completed may also be displayed. The book menu is organized in chapters (highest level menu items), comprising one or more pages (intermediate menu level), each comprising a plurality of challenges (lowest level menu items). Users can select challenges on a page by navigating with HA then selecting with EF and OC. Users can navigate between pages by selecting left/right arrow icons displayed on the left and right side of the page, using HA, EF and OC. Users can navigate between chapters by selecting a chapter icon (again using HA, EF and OC), which causes chapter tab marks to be selectable (where tab marks can again be selected with HA, EF and OC). The home scene may further comprise one or more objects that can be explored, such as a cabinet of potion (displaying completed potions), a history chest (displaying items won during gameplay by completing challenges), a closet (displaying items that can be selected by a user to configure their avatar, a door (to go back to the main menu), etc. Navigation between each of these objects and between items displayed after selection of these objects is performed using HA (for navigation) and EF+OC (for object selection).

The data from a game session may be stored locally on the device until the game is exited. The data may then be transmitted to a remote computer for further analysis. Alternatively, the data may be continuously or regularly (e.g. at regular interval, using a predetermined frequency of transmission of data packages) sent to a remote computer.

During gameplay, a user completes a series of challenges comprising making a particular potion. The user can select ingredients displayed on the screen to the left or right of a cauldron, using HA to the left or right side, respectively. The EF gesture can then be used to add the ingredient to the cauldron. The OC gesture can be used to return the ingredient to its previous position. The EF gesture can be used repeatedly to add multiple doses (e.g. pinches, drops, etc.) of an ingredient by raising and lowering the arm. The ingredient selected by EF may move to be above the cauldron, and may only return to its previous location upon detection of the OC gesture. For pourable ingredients, an item may be displayed indicating the amount to be poured, and a user may control the pouring using EF, lowering the arm causing pouring to start or increase, and raising the arm causing the pouring to slow down or stop. Pourable ingredients can be moved back to their previous location with OC after pouring is stopped. Addition of some ingredients may cause the temperature of the potion to show as having changed. A user may also be prompted to change the temperature of the potion that is being made, for example by an object being displayed (e.g. a thermometer displayed on the screen). A heating process may be started using HR to the right shoulder, and it may subsequently be stopped using HR to the left shoulder. The HR to the left may be made mandatory, such as e.g. by the potion overheating or the heating system breaking if not stopped (where “repair” requires the HR to the left). The temperature of a potion may be changed by adding one or more ingredients displayed as having an effect on temperature.

Challenges may be completed by adding all of a list of ingredients listed on a “scroll” displayed on the screen in displayed “amounts” (e.g. number of drops, pinches, amounts of liquid), and optionally while keeping the potion at a required temperature. In some cases, the potion may be shown as having an altered state (e.g. too thin, too thick, crystallizing, etc.) requiring the user to add a specific ingredient (e.g. starch, water, etc.) to bring the potion back to its normal state. Such ingredients may be selected and added in a similar manner to other ingredients. Points may be won by pre-empting and/or promptly dealing with altered states and temperature changes. A user may be prompted to stir the potion (for example by displaying an indicator that the potion density is excessive, or by the effect of some ingredients only appearing when the potion is stirred after addition of the ingredient, e.g. the effect of starch/water added to the potion may only be apparent when the potion is stirred). The user may be able to stir the potion using the TE gesture.

The HR gesture can be used to heat the potion, through the user turning their head to the right shoulder. This can start the heating process, which can be stopped by the user turning their head to the left. The heating process may be blocked for use if the user does not stop the heating process. The head rotation to the left may be used to unblock a blocked heating process.

Ingredients may be added as single items by selecting the ingredient using the EF gesture and dropping it in the cauldron using the OC gesture. Ingredients may be added as drops or pinches by selecting the ingredient using EF (or repeated EF for multiple drops/pinches). Ingredients may be poured (liquids or powders) into the cauldron using the EF gesture for selecting the ingredient then holding the arm down to keep pouring and lifting it up to stop pouring, then using OC to put away the ingredient.

A user may win points by completing potions, or correctly performing aspects of a potion completion task. Completing a potion may require that the user adds the correct ingredients listed on the user interface, in the listed amounts, while keeping the potion from overheating or becoming too dense (as indicated by indicators on the user interface).

The work demonstrated that an engaging exergaming therapeutic intervention development should involve diverse knowledge use partners to optimize its future usability and acceptability.

### Example 3: Feasibility of Turning Therapy into Fun with a Home-Based Exergame for Youth with Spinal Muscular Atrophy

The objective of this study was to assess the feasibility and usability of a fit-for-purpose exergaming intervention in youth (6-18 years old) with spinal muscular atrophy (SMA) and neurotypical controls.

The nature of how best to promote physical activity in youth with SMA is evolving. The use of active videogames for rehabilitation (exergaming) has a positive impact on motivation towards training, is flexible in scheduling, and has proven useful to enhance strength, coordination, and mobility in other conditions. The inventors have developed a home-based exergame (Tales from the Magic Keep™) specifically for youth with neuromuscular disorders using the Microsoft® Kinect Azure sensor.

A 4-week open label feasibility study was conducted across two Canadian sites. The exergaming intervention was used at home by participants at a target dose of 20 minutes four times a week. The Lasso informatics platform was used for data capture. Feasibility of the game was determined by assessing adherence, acceptability, and need for game adaptation. Adherence was quantified by the motion detector from the Azure Microsoft Kinect Platform and verified against a participant reported log. A Likert scale was used to evaluate the perceived value, experience, satisfaction and need for adaptation regarding the exergame across mental health motor function domains. Analysis was performed using intraclass correlation between the scores of items from the same domain to evaluate the agreement across participants. Usability was assessed using the System Usability Scale, and Cohen's Kappa was used to determine agreement between participants. Ten youth with SMA and five neurotypical controls were enrolled.

Tales from the Magic Keep™ was found to be acceptable and enjoyable to youth with a wide range of abilities. This novel technology intervention has the potential to improve access to physical activity in youth with SMA and other neuromuscular disorders.

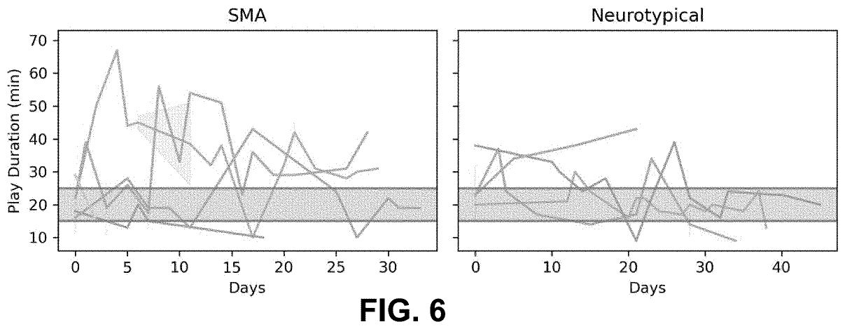

FIG. 6 shows the game play duration for each participant of the pilot study, for each day of the study, separated between two cohorts: a first cohort comprising 6 SMA patients and a second cohort comprising 4 neurotypical controls. The grey areas show required playing duration between 15 and 25 min). The data shows that participants used the digital physiotherapy tool as described herein and in particular that adherence to the prescribed regimen was good in the patient cohort. This indicates that the intervention is likely to achieve the primary endpoints of improving or reducing degradation in motor function and improving adherence to a physiotherapy regime.

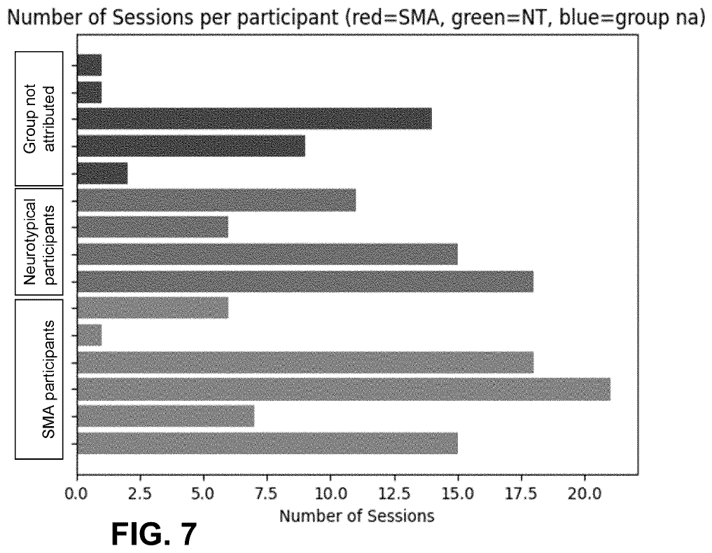

FIG. 7 shows the number of sessions played per participant, separated between neurotypical participants (middle set of bars) and SMA participants (red set of bars). The data show that many participants had a high number of sessions over the 4 weeks. Participants with a low amount of sessions could be due to (i) participants who dropped out during the study period; or (ii) supervisors who did a one-time test with the system.

Preliminary data analysis was performed using data from one SMA patient and one neurotypical control with many played sessions. In particular, the subject corresponding to the last bar in the neurotypical group (from the top) on FIG. 7 (number of played sessions: 18-subject 1 (NT)), and the subject corresponding to the fourth bar in the SMA group (from the top) on FIG. 7 (number of played sessions: 21-subject 2 (SMA)) were analysed. Session number 2 of each participant was used. This session has a length of: subject 1 (NT), session 2=23.74 min; subject 2 (SMA), session 2=27.50 min. During each session, raw data is collected. One session (one raw data file) contains the following parts: (i) Patient information (anonymized); (ii) Time; (iii) Calibration data; (iv) Kinect raw data; (v) Body joint data; (vi) 3D position; (vii) Orientation; (vii) confidence level (for the raw data); (viii) Additional information for each frame, e.g. which gestures has been achieved (a label or each frame indicating whether a gesture condition is achieved). The body joint data contains, for each position joint: position data (x, y, z coordinates), orientation data (x, y, z, w coordinates), and a confidence level (a score provided by the Kinect platform).

In addition, calculated data is also available for each session (labelsNames & labels, e.g. gesture conditions), which is used for gesture detection. This includes the following:
stageId—Stage ID (e.g. level in the game)
numberOfBodiesFound—Number of detected bodies from Kinect device. If the number is not equal 1, an error/warning message will be displayed.
bodyId—ID of the detected body
kinectTimestamp—Time of the Kinect device
systemTimestamp—Time of the system
unityTimestamp—Time of the Unity platform
OC_HandRightToHandTipRightRotation—Angle between finger vector and palm vector right side
OC_HandTipsToWristRightLength—Distance between hand tip to wrist right side
OC_HandLeftToHandTipLeftRotation—Angle between finger vector and palm vector left side
OC_HandTipsToWristLeftLength—Distance between hand tip to wrist left side
HA_RightArmForwardToSpineForwardRotation—Angle between torso forward and forearm vector right side
HA_ArmRightToDownRotation—Angle between torso down vector and upper arm vector right side
HA_ForearmRightToSpineUpRotation—Angle between torso up and forearm vector right side
HA_LeftArmForwardToSpineForwardRotation—Angle between torso forward and forearm vector left side
HA_ArmLeftToDownRotation—Angle between torso down vector and upper arm vector left side
HA_ForearmLeftToSpineUpRotation—Angle between torso up and forearm vector left side
EF_ArmRightToForeamrRightRotation—Angle between forearm vector and upper arm vector right side
EF ArmLeftToForeamrLeftRotation—Angle between forearm vector and upper arm vector left side
HR_HeadForwardToSpineForwardRotation—Angle between head forward vector and torso forward vector
HR_HeadForwardToSpineForwardRotation—Angle between head forward vector and torso forward vector
TE_LeftShoulderToRightShoulderRotation—Angle between left and right chest vectors
HA-L—Binary flag if gesture Horizontal Abduction Left is detected
HA-R—Binary flag if gesture Horizontal Abduction Right is detected
OC-L—Binary flag if gesture Open Close Left is detected
OC-R—Binary flag if gesture Open Close Right is detected
TE—Binary flag if gesture Thoracic Extension is detected
HR-L—Binary flag if gesture Head Rotation Left is detected
HR-R—Binary flag if gesture Head Rotation Right is detected
EF-L—Binary flag if gesture Elbow Flexion Left is detected
EF-R—Binary flag if gesture Elbow Flexion Right is detected.

As explained above, the following five gestures (physiotherapy exercises) are implemented: (i) Elbow flexion; (ii) Thoracic extension; (iii) Head rotation; (iv) Horizontal abduction; (v) Open-Close hand. The Kinect platform reports joint information for 32 joints, indexed from 0 to 31 (see learn.microsoft.com/en-us/azure/kinect-dk/body-joints). The following joints are used for the gesture detection: left elbow, right elbow, left wrist, right wrist, left shoulder, right shoulder, head, neck, spine at chest level, spine at navel level, pelvis, left clavicle, right clavicle, left hand, right hand, left hand tip, and right hand tip. The data of the other body joints are ignored, but still recorded.

As explained above, the elbow flexion and horizontal abduction are single gestures, whereas the head rotation, thoracic extension and open-close hand are sequential gestures. Single gestures have one condition which must be fulfilled, sequential gestures have multiple conditions to be fulfilled. In the raw data, the data which is used for the gesture (pt exercises) detection is recorded in each frame. The Microsoft Kinect device will record 30 frames per second. Detection of each gesture in this example is explained below.

### Open-Close Hand:

body joints: vector 1: wrist, hand; vector 2: hand, hand tip; distance 1: wrist, hand tip; elbow flexion angle (see elbow flexion gesture)
rules: sequence #1 (closed hand): threshold: 35 degrees, range: 30-100 degrees (angle between vectors 1 and 2); sequence #2 (contains 3 conditions): #condition 1: (open hand): threshold: 40 degrees, range: 100-180 degrees (angle between vectors 1 and 2); condition #2 (distance): the ratio of the maximum distance and the current distance needs to be 0.375 (subject to calibration); condition #3 (elbow flexion): elbow needs to be flexed.
data: open-close hand is a sequential gesture. In the data file there are 2 data points about this gesture in each data frame. This is: OC_HandRightToHandTipRightRotation (angle value) and OC_HandTipsToWristRightLength (distance value); OC-R (boolean value if the gesture has been achieved).

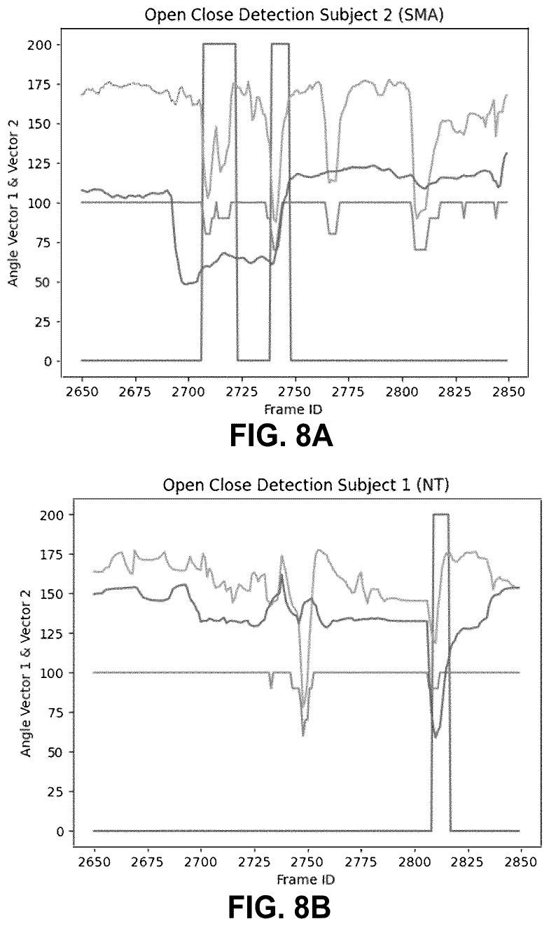

FIGS. 8A-8B show examples of the open-close gesture detection in subject 2 (SMA) and subject 1 (NT). The figures show the angle between vector 1 and vector 2 in each frame (orange line), the distance between hand tip and wrist in each frame (green line, upscaled—multiplied by 100), the angle between the forearm vector and upper arm vector (red line, elbow flexion), and a Boolean indicator of detection of the gesture. The data show that an open-close gesture is identified when the elbow is flexed and the angle between vectors 1 and 2 decreases then increases within the range above.

### Elbow Flexion:

body joints: Vector 1 (Forearm vector): Elbow, Wrist; Vector 2 (Upper arm vector): Elbow, Shoulder.
rules: Range: 30-100 degrees (angle between vectors 1 and 2) (initial angle 65 degrees, threshold 35 degrees, i.e. angle in range 65±35 degrees)
data: Elbow flexion is a single gesture. The calculated angle (as described above) as well as the information if the gesture is fulfilled for each frame is available in the raw data file. The information is stored under the labels: EF_ArmRightToForeamrRightRotation (angle value, right side); EF-R (boolean value if the gesture has been achieved, right side); EF_ArmLeftToForeamrLeftRotation (angle value, left side); EF-L (boolean value if the gesture has been achieved, left side).

The gesture is detected whenever the angle between vectors 1 and 2 drops under a specified value, which is a value within the above range identified through personal calibration.

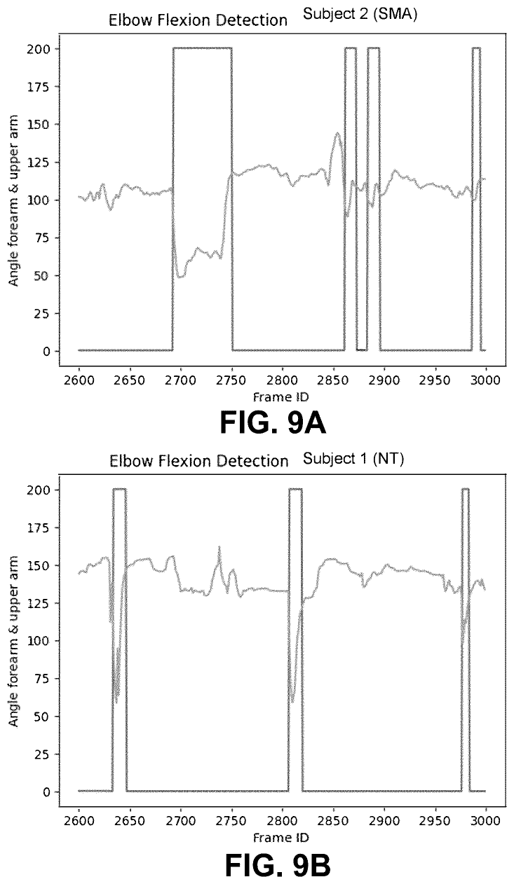

FIGS. 9A-9B show examples of the elbow flexion gesture detection in subject 2 (SMA) and subject 1 (NT). Both figures show the angle between vectors 1 and 2 for each frame, and a Boolean indicator of detection of the elbow flexion gesture. In FIG. 9A, elbow flexion is detected 4 times. Whenever the angle drops under a specified value (gesture condition+calibration value), the gesture movement is fulfilled. In FIG. 9B, elbow flexion is detected 3 times. Whenever the angle drops under a specified value (gesture condition+calibration value), the gesture movement is fulfilled.

### Thoracic Extension:

body joints: Vector 1: Clavicle, Shoulder (both sides); Vector 2: Neck, Elbow (both sides). Vectors 1 and 2 on the left side are used to calculate a left chest vector. Vectors 1 and 2 on the right side are used to calculate a right chest vector. The rules apply to the angle between the left and right chest vectors.
rules: Sequence #1: Angle: 190 degrees, Threshold: 12 degrees; Sequence #2: Angle: 160 degrees, Threshold: 15 degrees.
data: Thoracic extension is a sequential gesture. The calculated angle (as described above) as well as the information if the gesture is fulfilled for each frame is available in the raw data file. The information is stored under the labels: TE_LeftShoulderToRightShoulderRotation (angle value); TE (boolean value if the gesture has been achieved).

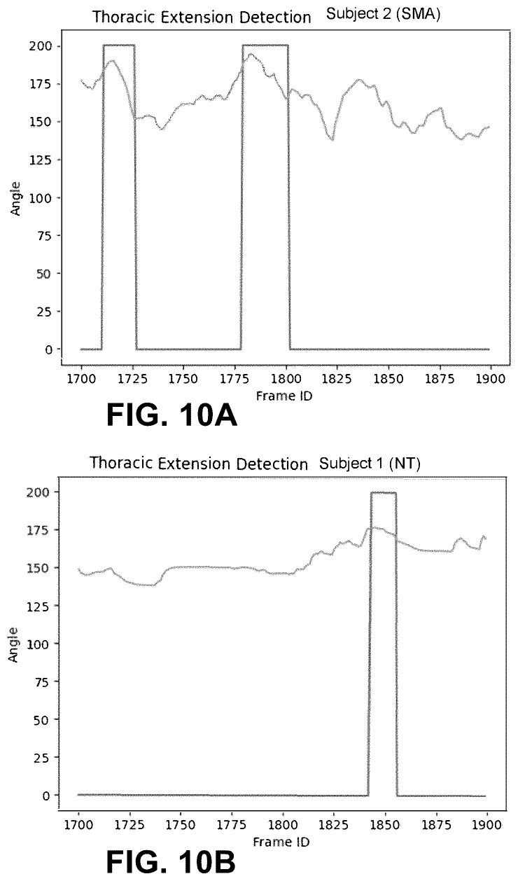

FIGS. 10A-10B show examples of the thoracic extension gesture detection in subject 2 (SMA) and subject 1 (NT). Both figures show the chest angle, and a Boolean indicator of detection of the thoracic extension gesture. In FIG. 10A, thoracic extension is detected 2 times. Whenever the angle fulfills both sequences (when taking into account the gesture condition and calibration value), the gesture movement is fulfilled. In FIG. 10B, thoracic extension is detected 1 time. Whenever the angle fulfills both sequences (when taking into account the gesture condition and calibration value), the gesture movement is fulfilled.

### Head Rotation:

body joints: Vector 1 (head forward): Head; Vector 2 (torso forward): Neck, Spine_Chest, Spine_Naval, Pelvis.
rules: Sequence #1: Angle: 42 (right rotation),-42 (left rotation), Threshold: 5; Sequence #2: Angle: 0, Threshold: 10.
data: Head rotation is a sequential gesture. The calculated angle (as described above)) as well as the information if the gesture is fulfilled for each frame is available in the raw data file. The information is stored under the labels: HR_HeadForwardToSpineForwardRotation (angle value, both sides); HR-R (boolean value if the gesture has been achieved, right side); HR-L (boolean value if the gesture has been achieved, left side).

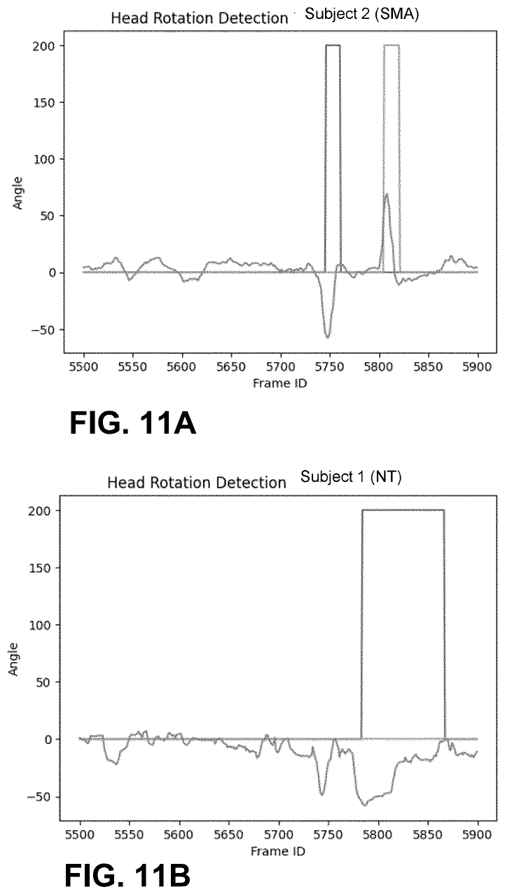

FIGS. 11A-11B show examples of the head rotation gesture detection in subject 2 (SMA) and subject 1 (NT). Both figures show the angle between vectors 1 and 2 for each frame, and a Boolean indicator of detection of the head rotation gesture (different colour for left and right). In FIG. 11A, head rotation is detected 2 times (one left, one right). Whenever the angle fulfills both sequences (when taking into account the gesture condition and calibration value), the gesture movement is fulfilled. In FIG. 11B, head rotation is detected 1 time (left). Whenever the angle fulfills both sequences (when taking into account the gesture condition and calibration value), the gesture movement is fulfilled.

### Horizontal Abduction:

body joints: Vector 1 (arm): Vector 2 (torso forward): Neck, Spine_Chest, Spine_Naval, Pelvis; Vector 3 (torso down); Vector 4 (upper arm): Vector 5 (torso up vector): Vector 6 (forearm vector):
rules: Condition #1: Angle (Vector 1 & Vector 2): 35 degrees, Threshold: 35 degrees, Range: 0-70 degrees; Condition #2: Angle (Vector 3 & Vector 4): 55 degrees, Threshold: 25 degrees, Range: 30-80 degrees; Condition #3: Angle (Vector 5 & Vector 6): 95 degrees, Threshold 18 degrees, Range: 77-113 degrees
data: Horizontal abduction is a sequential gesture. The calculated angles (as described above) as well as the information if the gesture is fulfilled for each frame is available in the raw data file. The information is stored under the labels: HA_RightArmForwardToSpineForwardRotation (angle value); HA_ArmRightToDownRotation (angle value); HA_ForearmRightToSpineUpRotation (angle value); HA_LeftArmForwardToSpineForwardRotation (angle value); HA_ArmLeftToDownRotation (angle value); HA_ForearmLeftToSpineUpRotation (angle value); HA-R (boolean value if the gesture right side has been achieved); HA-L (boolean value if the gesture left side has been achieved).

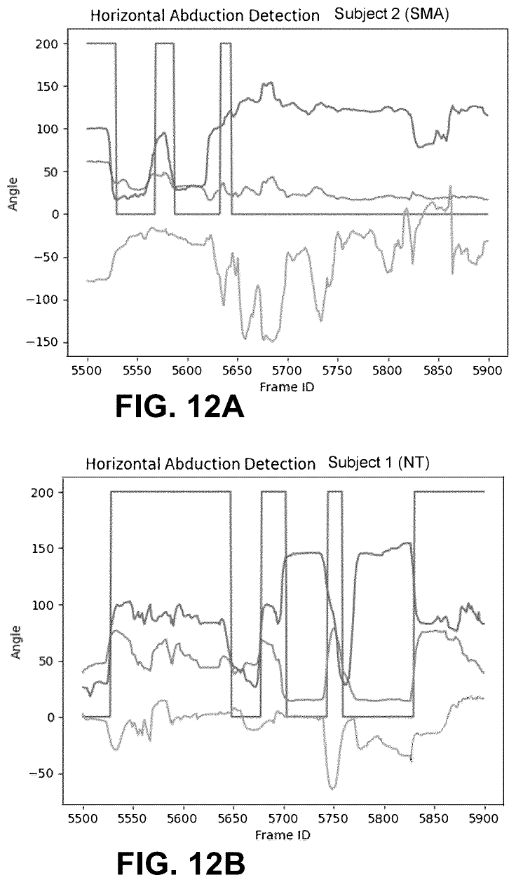

FIGS. 12A-12B show examples of the horizontal abduction gesture detection in subject 2 (SMA) and subject 1 (NT). Both figures show: (i) the angle between vectors 1 and 2 for each frame (bottom line), (ii) the angle between vectors 3 and 4 for each frame (middle line), (iii) the angle between vectors 5 and 6 for each frame (top line), and (iv) a Boolean indicator of detection of the horizontal abduction gesture. In FIG. 12A, horizontal abduction is detected 3 times. In FIG. 12B, horizontal abduction is detected 4 times. The figures also show the effect of calibration, where the values of the angles between vectors 3-4 and 5-6 reached in FIG. 12A to trigger detection of the gesture (SMA subject) are lower than in FIG. 12B (NT subject), and the angle between vectors 1 and 2 is more variable in the SMA patient data (FIG. 12A).

Collectively, the data from this feasibility study demonstrate that participants used the digital physiotherapy tool as described herein, that the gestures were performed and detected as expected including by SMA patients, and further show the benefits of personal calibration of the specific thresholds used in the gesture detection rules.

### REFERENCES

Chen X., Siebourg-Polster J., Wolf D., Czech C., Bonati U., Fischer D., Khwaja O., Strahm M. (2017) Feasibility of Using Microsoft Kinect to Assess Upper Limb Movements in Type III Spinal Muscular Atrophy Patients. PLSS ONE 12 (1): e0170472.
Main M., Kairon H., Mercuri E., Muntoni F. The Hammersmith functional motor scale for children with spinal muscular atrophy: a scale to test ability and monitor progress in children with limited ambulation. Eir J Paediatr Neurol. 2003; 7 (4): 155-9.
Krosschell K J, Maczulski J A, Crawford T O, Scott C, Swoboda K J. A modified Hammersmith functional motor scale for use in multi-center research on spinal muscular atrophy. Neuromuscul Disord. 2006 July; 16 (7): 417-26.
Berard C., Payan C., Hodgkinson I., Fermanian J., Group MFMCS. A motor function measure for neuromuscular diseases. Construction and validation study. Neuromuscul Disor. 2005; 15 (7): 463-70.
Blaschek A, Hesse N, Warken B, Vill K, Well T, Hodek C, Heinen F, Müller-Felber W, Schroeder A S. Quantitative Motion Measurements Based on Markerless 3D Full-Body Tracking in Children with SMA Highly Correlate with Standardized Motor Assessments. J Neuromuscul Dis. 2022; 9 (1): 121-128.
Lowes, L. P., Alfano, L. N., Crawfis, R., Berry, K., Yin, H., Dvorchik, I., Flanigan, K. M. and Mendell, J. R. (2015), Reliability and validity of active-seated: An outcome in dystrophinopathy. Muscle Nerve, 52:356-362.
Law M, Baptiste S, McColl M, Opzoomer A, Polatajko H, Pollock N. The Canadian occupational performance measure: an outcome measure for occupational therapy. Can J Occup Ther. 1990 April; 57 (2): 82-7.
E. Gargaun, A. Seferian, G. Quicke et al. Innovative home activity monitoring in non-ambulant patients with spinal muscular atrophy: a multicenter observational trial. Neuromuscular disorders, vol. 27, suppl. 2, S225, 2017.
King, G., Law, M., King, S., Hurley, P., Hanna, S., Kertoy, M., Rosenbaum, P., & Young, N. (2004). Children's Assessment of Participation and Enjoyment (CAPE) and Preferences for Activities of Children (PAC). San Antonio, T X: Harcourt Assessment, Inc.
Trundell D, Skalicky A, Staunton H, Hareendran A, Le Scouiller S, Barrett L, Cooper O, Gorni K, Seabrook T, Jethwa S, Cano S. Development of the SMA independence scale-upper limb module (SMAIS-ULM): A novel scale for individuals with Type 2 and non-ambulant Type 3 SMA. J Neurol Sci. 2022 Jan. 15; 432:120059.
Lott, D J, Taivassalo, T, Cooke, K D, et al. Safety, feasibility, and efficacy of strengthening exercise in Duchenne muscular dystrophy. Muscle & Nerve. 2021; 63:320-326.

1. A computer-implemented method comprising:
providing a user interface to a subject that has been diagnosed as having a neuromuscular disease or disorder and optionally undergoing treatment with a therapeutic compound or composition for the treatment of the neuromuscular disease or disorder;
receiving movement data comprising coordinates of a plurality of joints of the subject or data derived therefrom, at a plurality of time points, and
in response to receiving movement data satisfying a set of predetermined criteria, triggering a change in the user interface, wherein the change is specific to the set of predetermined criteria satisfied and wherein the set of predetermined criteria is selected from a plurality of sets of predetermined criteria, each set identifying a predetermined movement.

2. The method of claim 1, further comprising: upon receiving the movement data, determining whether the movement data satisfies each of the plurality of sets of predetermined criteria.

3. The method of any preceding claim, wherein the plurality of sets of predetermined criteria comprise: a first set of predetermined criteria identifying an elbow flexion gesture, a second set of predetermined criteria identifying a horizontal abduction gesture, a third set of predetermined criteria identifying a head rotation gesture, a fourth set of predetermined criteria identifying a thoracic extension gesture, and a fifth set of predetermined criteria identifying an open-close hand gesture.

4. The method of any preceding claim, wherein one or more of the predetermined criteria apply to angles between predetermined vectors derived from one or more joints or to projections of angles between predetermined vectors on predetermined body planes and/or wherein each set of predetermined criteria is individually selected from: (i) a set of criteria that apply to movement data at a single time point and (ii) a set of criteria comprising at least one criterion that applies at a first time point and at least one criterion that applies at a second time point.

5. The method of claim 4, wherein the predetermined vectors are selected from: a forearm vector, an upper arm vector, a chest vector, a head forward vector, a torso forward vector, an arm vector, a torso down vector, a torso up vector, a finger vector, and a palm vector.

6. The method of any preceding claim, wherein at least one criterion applies to an angle between two predetermined vectors derived from one or more joints or to the projection of an angle between two predetermined vectors on a predetermined body plane, and wherein the method comprises calibrating the at least one criterion by recording movement data of the subject while the subject is performing the movement associated with the predetermined criterion and identifying an expected range of the angle or the angle projection while the subject is performing the movement associated with the predetermined criterion.

7. The method of any preceding claim, wherein the plurality of sets of predetermined criteria comprise one or more of:
a. a first set of predetermined criteria identifying an elbow flexion gesture comprises a criterion that the angle between a forearm vector and an upper arm vector on the same side: changes from a first value in a first predetermined range to a second value in a second predetermined range, or is below a threshold value that is in the second predetermined range, optionally wherein the first predetermined range is between 80 degrees and 120 degrees and the second predetermined range is between 10 degrees and 50 degrees;
b. a second set of predetermined criteria identifying a horizontal abduction gesture comprises a first predetermined criterion that the angle between a torso forward vector and an arm vector is within a first predetermined range or below a first predetermined threshold value that is in said first predetermined range, at a first time point, and a second predetermined criterion that the angle between the torso forward vector and the arm vector is within a second predetermined range or above a second predetermined threshold value that is in the second predetermined range, at a second time point that is subsequent to the first time point, optionally wherein the first predetermined range is between 0 and 70 degrees and the second predetermined range is between 30 degrees and 80 degrees, and/or wherein a second set of predetermined criteria identifying a horizontal abduction gesture further comprises a third predetermined criterion that an angle between a torso up vector and a forearm arm vector on the same side as that to which the first predetermined criterion applies is within a predetermined range at the first and/or second time points;
c. a third set of predetermined criteria identifying a head rotation gesture comprises a first predetermined criterion that the angle between a head forward vector and a torso forward vector at a first time point is within a first predetermined range or above a first predetermined threshold that is in said first predetermined range, and a second predetermined criterion that the angle between the head forward vector and the torso forward vector is within a second predetermined range or below a second predetermined threshold that is in said second predetermined range, at a second time point that is subsequent to the first time point, optionally wherein the first predetermined range is 42±5 degrees and the second predetermined range is 0±10 degrees;
d. a fourth set of predetermined criteria identifying a thoracic extension gesture comprises a first predetermined criterion that the angle between the left and right chest vectors at a first time point is within a first predetermined range or above a predetermined threshold value that is in the first predetermined range, and a second predetermined criterion that the angle between the left and right chest vectors is within a second predetermined range at a second time point that is subsequent to the first time point, optionally wherein the first predetermined range is 190±12 degrees and the second predetermined range is 160±15 degrees; and/or
e. a fifth set of predetermined criteria identifying an open-close hand gesture comprises: a first predetermined criterion that an angle between a finger vector and a palm vector on the same side at a first time point is within a first predetermined range or below a first predetermined threshold value that is in the first predetermined range, and a second predetermined criterion that the angle between the finger vector and a palm vector is within a second predetermined range or above a second predetermined threshold value that is in the second predetermined range, at a second time point that is subsequent to the first time point, optionally wherein the first predetermined range is between 30 degrees and 100 degrees and the second predetermined range is between 100 degrees and 180 degrees, and/or wherein a fifth set of predetermined criteria identifying an open-close hand gesture further comprises a third predetermined criterion that applies to an angle between a forearm vector and an upper arm vector on the same side as that which satisfies the first and second predetermined criteria, optionally wherein the third predetermined criterion is defined as the angle changing between a first predetermined value and a second predetermined value for identifying an elbow flexion, or wherein the third predetermined criterion is defined as the angle being at or below the second predetermined value for identifying an elbow flexion, and/or wherein a fifth set of predetermined criteria identifying an open-close hand gesture further comprises a fourth predetermined criterion that applies to the distance between a hand tip joint and a wrist joint on the same side as that which satisfies the first and second predetermined criteria, optionally wherein the fourth predetermined criterion is defined as said distance being less than a predetermined ratio of a reference value for said distance.

8. The method of any preceding claim, wherein the plurality of joints include one or more or all of a plurality of locations that aligns with a subject's: left elbow, right elbow, left wrist, right wrist, left shoulder, right shoulder, head, neck, spine at chest level, spine at navel level, pelvis, left clavicle, right clavicle, left hand, right hand, left hand tip, and right hand tip.

9. A therapeutic compound or composition for use in a method of treating a neuromuscular disease or disorder in a subject, wherein the method comprises:
administering a therapeutically effective amount of the therapeutic compound or composition to the subject, and
implementing a digital physiotherapy method comprising the steps of any of claims 1 to 8;
wherein the method improves, stabilises or reduces the degradation in a motor function score in the subject.

10. The therapeutic compound or composition for use according to claim 9, wherein the method comprises implementing the digital physiotherapy method according to a predetermined digital physiotherapy dosage regimen, wherein the predetermined physiotherapy dosage regimen comprises the use of the digital physiotherapy tool for at least 15 to 25 minutes per day at least 4 times per week.

11. The therapeutic compound or composition for use according to claim 9 or claim 10, wherein the method increases the likelihood of continued administration of a physiotherapy dosage regimen, wherein continued administration of a physiotherapy dosage regimen refers to adherence to the physiotherapy dosage regimen for at least 3 months, at least 6 months, at least 9 months or at least 12 months.

12. The therapeutic compound or composition for use according to any of claims 9 to 11, wherein the motor function score is selected from: the revised upper limb module (RULM), the Hammersmith Functional Motor Scale (HFMS), the Motor Function Measure (MFM), the Fatigue Severity Scale (FSS), the Modified Fatigue Impact Scale (MFIS), the Visual Analogue Scale for Fatigue (VAS-F), the PROMIS-SF, the SMAIS, the SMAIS-ULM, the Children's Assessment of Participation and Enjoyment (CAPE), the PedsQL™ 4.0 Generic Core Scales (Pediatric Quality of Life Inventory™), and the Canadian Occupational Performance Measure (COPM), and/or wherein the method improves, stabilises or reduces a degradation in a motor function score after about 8 weeks, about 12 weeks or about 16 weeks of treatment, compared to a subject treated with a method comprising administering a therapeutically effective amount of the therapeutic compound or composition to the subject without implementing the digital physiotherapy method, and/or wherein the therapeutic compound or composition is a disease modifying therapeutic, optionally wherein the therapeutic compound or composition is selected from nusinersen and risdiplam.

13. The method of any preceding claim, wherein the subject has a neuromuscular disease or disorder selected from: a motor neuron disease, a toxic neuropathy, a congenital myopathy, a muscular dystrophy, and spinal muscular atrophy, optionally wherein the spinal muscular atrophy is type II or type III SMA, wherein the neuromuscular junction disorder is myasthenia gravis or Lambert-Eaton Syndrome, wherein the muscular dystrophy is selected from: Duchenne muscular dystrophy, Becker muscular dystrophy, congenital muscular dystrophy, and facioscapulohumeral muscular dystrophy, or wherein the motor neuron disease is amyotrophic lateral sclerosis, preferably wherein the subject has type II or type III SMA.

14. A system comprising:
at least one processor; and at least one non-transitory computer readable medium containing instructions that, when executed by the at least one processor, cause the at least one processor to perform a digital physiotherapy method comprising the steps of:
providing a user interface to a subject that has been diagnosed as having a neuromuscular disease or disorder and optionally undergoing treatment with a therapeutic compound or composition for the treatment of the neuromuscular disease or disorder;
receiving movement data comprising coordinates of a plurality of joints of the subject or data derived therefrom, at a plurality of time points, and
in response to receiving movement data satisfying a set of predetermined criteria, triggering a change in the user interface, wherein the change is specific to the set of predetermined criteria satisfied and wherein the set of predetermined criteria is selected from a plurality of sets of predetermined criteria, each set identifying a predetermined movement;
optionally wherein the system further comprises a display device operably coupled to the processor for display of the user interface, and a movement sensing device operably coupled to the process for acquisition of the movement data; and/or
wherein the instructions further cause the processor to perform the steps of any of claims 2 to 8.

15. A method of improving, stabilising or reducing the degradation in a motor function score in a subject undergoing treatment with a therapeutic compound or composition for treating a neuromuscular disease or disorder, and/or increasing the likelihood of continued administration of a physiotherapy dosage regimen, wherein continued administration of a physiotherapy dosage regimen refers to adherence to the physiotherapy dosage regimen for at least 3 months, at least 6 months, at least 9 months or at least 12 months, wherein the method comprises implementing a computer-implemented method for digital physiotherapy comprising the steps of:
providing a user interface to the subject;
receiving movement data comprising coordinates of a plurality of joints of the subject or data derived therefrom, at a plurality of time points, and
in response to receiving movement data satisfying a set of predetermined criteria, triggering a change in the user interface, wherein the change is specific to the set of predetermined criteria satisfied and wherein the set of predetermined criteria is selected from a plurality of sets of predetermined criteria, each set identifying a predetermined movement.

16. The method of claim 15, wherein the method has the features of any of claims 1 to 12.
